(19) United States
(12) Patent Application Publication
(10) Pub. No.: US 2023/0414129 A1
(43) Pub. Date: Dec. 28, 2023

(54) SYSTEM AND METHOD FOR MONITORING INDIVIDUAL'S DAILY ACTIVITY

(71) Applicant: ZE CORRACTIONS LTD., Mevasseret Zion (IL)

(72) Inventor: Eldad Izhak HOCHMAN, Raanana (IL)

(73) Assignee: ZE CORRACTIONS LTD., Mevasseret Zion (IL)

(21) Appl. No.: 18/037,055

(22) PCT Filed: Dec. 9, 2021
(86) PCT No.: PCT/IL2021/051464

§ 371 Date: May 15, 2023

Related U.S. Application Data
(60) Provisional application No. 63/123,040, filed on Dec. 9, 2020.

(51) Int. Cl. A61B 5/11

(52) U.S. Cl. CPC A61B 5/1118

(57) ABSTRACT

A monitoring system and method are presented for monitoring an individual's activity. The monitoring system comprises a control system configured as a computer system, and being configured and operable to be responsive to input data comprising sensing data collected over time from at least a part of individual's body by one or more sensors of predetermined one or more types and being indicative of a motion pattern characterizing a certain activity of the individual, to process the input data and generate output data indicative of a cognitive error detection by the individual in said activity characterizing a cognitive operational state of the individual during said activity.

### TECHNOLOGICAL FIELD AND BACKGROUND

The present invention is in the field of monitoring decision-making of individuals with regard to their activities, and relates to a method and system for detection of kinematic errors in the activity of an individual.

Decision-making is a cognitive process resulting in the mindful or unconscious selection from a variety of possible alternatives, resulting in a selected choice that in some cases might cause an immediate action, which may be associated with individual's interaction with/operation of a certain external device/machine. Incorrect decisions (or incorrect actions resulting from such decisions) can result from internal cognitive errors made during the decision-making process, and may be associated with a variety of reasons/conditions; as well as can include decisions or actions which may have been initially correct but have become incorrect during or shortly after they were made. Incorrect decisions which are made in human-machine interaction scenarios are often followed by reactions which result in undesirable outcomes, which may have in some cases damaging and even devastating consequences. For example, when using a computerized device, such as a personal computer, a tablet or a smart phone, it is not uncommon that users perform erroneous actions such as pressing incorrect button. When operating industrial instruments or machines, incorrect decisions may lead to incorrect actions which in some cases may result in injury or even death.

U.S. Pat. No. 10,413,246 discloses the technique developed by the inventors of the present application for detection of an interaction-error. The interaction-error is derived from an incorrect decision and is directed to interacting with a machine. According to this technique, during human-machine interaction, command related data values are obtained, which characterize any one of an interacting-command and an interacting-action. The command related data values are compared with command related reference data values, and an interaction-error is identified if a difference between the command related data values and the command related reference data values complies with a predefined criterion.

### GENERAL DESCRIPTION

There is a need in the art for a novel technique for monitoring individual's operational state (i.e., cognitive, emotional, physiological etc.) and/or quality of motor functioning, and detecting kinematic errors in individual's daily activity resulting from incorrect cognitive decision-making, irrespective of whether such activity is associated with individual's interaction with an external object or not.

In individual's daily activity scenarios, decisions made by the individual often result in one or more cognitive commands intended to generate a specific action, produced in reaction to observed operation of a certain device affected by the individual's activity or generated independently of any device operation. Brain commands generated in such scenarios include commands to various parts of different systems of the individual's body which occur in response to the performance of the individual's activity. The individual's activity can be either individual's interaction with a device which has a direct effect on the device operation or activity which does not have a direct effect on the device operation (e.g. activity involving only the observation of the operation of a device and/or activity executed independently of device operation however, monitored by the device).

Motion command data generated by individual's brain includes commands of the type instructing a body part to perform one or more actions that may or may not be directed for controlling a device for performing a desired operation. Such actions include conscious actions and non conscious actions, as well as discrete actions and continuous actions. Motion command data may also include commands which do not result in a physical reaction of a body part. For example, these include commands of the type providing direct communication pathway between the brain of an individual and an external device and enables the individual to control the external device by cognitive commands intended to initiate some operation of the device without physically interacting with the device.

Motion command data of the type instructing a body part to perform one or more actions that may not be directed for controlling a device for performing a desired operation may also include commands where the person monitors the activity of a machine and develops an expectation about machines actions. When the machine deviates from the expectation of the person, the person's brain detects an error in the activities of the machine because its activity does not match the person's expectations.

As explained above, incorrect decisions are often followed by reactions which may result in undesirable outcomes. An individual's mind is capable of identifying incorrect decisions, before the individual is consciously aware of the error. In response to identification of an incorrect decision and/or a related erroneous command, the individual mind compensates for it (e.g. inhibits/cancels and/or replaces/corrects the error) in attempt to reverse the decision and avoid the execution of the erroneous command or at least reduce its effect. Alternatively, the individual mind may compensate for the error by reducing the error force initiating a counter movement/response or initiating a secondary corrective response.

Detection of incorrect decision and/or actions, compensation, cancellation and/or correction attempts are reflected by a mental process revealed by increased activity in brain regions associated with error detection, reduced activity in brain regions associated with the unwanted event and/or increased activity in brain regions associated with the new event. Because these error-related mental processes are eventually aimed at canceling, correcting or compensating for the incorrect command, they immediately affect brain regions associated with motor planning and execution. Activity in brain motor regions immediately affects bodily muscles and slightly later, involve error-related changes in bodily systems such as reactions of the autonomic nervous system (e.g. detectible changes to pupil dilation, heart beats, galvanic skin response, breath). These autonomic nervous system reactions to brain error detection may in turn result in additional error-related motor reactions.

In the description below, all error-detection related processes (e.g., error detection, cancellation, inhibition, correction, compensation) are referred to for simplicity as error detection.

As a result of such error detection-related reactions, measurable parameters (command data or motion command data), which characterize an erroneous command and/or a resulting erroneous action are different than those of a correct command and/or resulting correct action.

As described above, incorrect decisions or actions may occur when an individual does not operate any device but rather initiates an action aimed at completing a certain task or reaching a certain goal (i.e., shoe tying, grasping a glass of water, standing up) or operates a non-electronic device (i.e., tennis racket). Here errors may involve for example, undershooting, overshooting, exerting too much or too little pressure and so forth.

In addition to the above-described examples of decision-making situations occurring in individual's daily activity which might result in incorrect decisions (or incorrect actions resulting from such decisions), these can also include instances where movement progress deviates from the initial movement plan or goal or needs to be changed according to an updated movement plan or updated goal. Deviations may occur during well-defined discrete events, such as button presses or fast reaching movements or during continuous and not easily parsed motions such as shoe tying.

The present invention provides a novel technique for quantification of individual's operational state (e.g., low motivation, cognitive load, intoxication, mental fatigue, drowsiness, stress, inattention, vertigo, motion sickness) according to cognitive error detection by said individual in his/her activity. This is implemented in the invention using a sensing system in signal/data communication with a control system. The sensing system may be of any known suitable type including one or more sensors capable of monitoring movement intention or movement of at least a portion of the individual's body and providing corresponding motion pattern data. The control system analyses the motion pattern data to identify a motion profile indicative of the individual's cognitive operational state according to individual's error detection, prior, during or after to actual occurrence of the error.

As described above, the individual's activity to be monitored/controlled may not be associated with individual's interaction with any electronic device, but rather may be activity aimed at completing a certain task or reaching a certain goal or operation of a non-electronic device, and the indication is thus received from the individual's own movements. Although, in cases where the individual is interacting with a device (electronic device), the indication may be received from that device.

The data processing technique of the present invention is aimed at determining, in the measured motion pattern data, a motion profile indicative of the individual's cognitive operational state according to individual's error detection. To this end, the motion pattern data is analyzed to determine the movement progress deviation from the initial movement plan or goal (such deviations may occur during well-defined discrete events or during continuous and not easily parsed motions), identify and analyze different types of deviations indicating error detection in the individual's brain, and compare the measured error detection related deviations with predefined (stored) reference error detection related deviations, in order to determine whether the measured error related deviation and/or a relation (e.g. difference value) between the measured and reference error related deviations, indicate a change in the individual's cognitive operational state.

In some embodiments, the data processing and analyzing technique is as follows. The sensed/measured motion data (motion pattern), collected over time, is analyzed to determine whether the movement of a relevant part of individual's body during certain activity can be classified as a goal directed movement, and select the motion pattern corresponding to the goal directed movement for further processing. Such processing includes searching for predetermined selected segment(s)/time slot(s) of the motion pattern to identify whether they include/are indicative of error-related movements/patterns. The predetermined segments of the motion pattern to be selected include a motion pattern segment from the first instant of the goal-directed movement until after the first instant of the latest motion pattern that served the decision of goal-directed movement.

Throughout this description the terms “goal-directed movement” and “purposeful movement” are used interchangeably and describe the same thing. Cognition plays a major role in purposeful movements, all of which are goal-directed. Purposeful movements (goal-directed movements) belong to voluntary movements (e.g., while driving a car, volitionally reaching the brakes) or well-learned movements that were initially voluntary and after training became automatic (e.g., while seated next to the car driver, performing a brake-reaching motion in response to the brake lights of the vehicle in front of us), as opposed to unlearned reflexes (e.g., automatic closing of the eyes when tired, reaching back when accidentally touching a hot glass), and are initiated to accomplish a specific goal, e.g. gestures involving objects (tools) or not involving objects, e.g. waving hello.

The reaction times (the time between the presentation of a stimulus and the initiation of a voluntary response) of purposeful movement are usually significantly longer than the latencies of reflex responses elicited by comparable stimuli. The successful performance of a goal-directed movement task requires a complex interplay of many cognitive skills interacting with sensory, perceptual, emotional and motor skills.

The error-related motion patterns are indicative of error-related motion command data originated in the individual's brain. This error-related motion command data is analyzed in relation to corresponding motion command error-related reference data (i.e., error-related reference motion patterns occurring in a similar movement, e.g. similar limb, kinematics, force, distance traveled, direction, movement goal, error size, context etc.). Based on this data analysis, the control system generates data regarding the individual's operational state.

Thus, according to a broad aspect of the invention, there is provided a monitoring system for monitoring an individual's activity, the monitoring system comprising a control system configured as a computer system comprising data input and output utilities, a memory, and a data processor and analyzer, the control system being configured and operable to be responsive to input data comprising sensing data collected over time from at least a part of individual's body by one or more sensors of predetermined one or more types and being indicative of a motion pattern characterizing a certain activity of the individual, to process said input data by applying thereto one or more machine learning models and generate output data indicative of a cognitive error detection by said individual in said activity characterizing a cognitive operational state of the individual during said activity.

The control system may be configured and operable for data communication with one or more measured data providers to receive, from each measured data provider, the input data comprising the motion patterns.

The input data includes sensing data which may comprise the motion patterns measured over time and being indicative of movement intention or movement of at least a part of the individual's body during said activity, and/or may comprise the motion patterns measured over time on a device operated by the individual during said activity.

The control system may be configured and operable to carry out the following: analyze the motion patterns to determine movement progress deviation from an initial movement goal; identify and analyze different types of deviations indicating error detection in the individual's brain; and compare identified error detection related deviations to predefined reference error detection related deviations, to thereby determine whether at least one of the identified error related deviations and a relation between identified and reference error related deviations is indicative of a change in the individual's cognitive operational state.

In some embodiments, the control system comprises: an analyzer configured and operable to analyze the input data, and, upon identifying that the motion patterns comprise a pattern corresponding to a goal directed movement performed by the individual during said activity, generating corresponding decision data; and an error identifier utility configured and operable to identify at least one predetermined segment in the goal directed movement pattern, and process said at least one predetermined segment, and, upon determining that motion profile of said at least one predetermined segment is indicative of movements cognitively recognizable by the individual as error-related movements, generate output data indicative of error-related motion pattern enabling evaluation of the operational state of the individual.

The at least one predetermined segment of the motion pattern may include motion pattern from a first instant of the goal-directed movement until after a first instant of a latest motion pattern that served the decision of the goal-directed movement.

In some embodiments, the analyzer is configured and operable to analyze movement kinematic data derived from said motion patterns to determine differentiation between movements that can be classified as goal directed and non goal directed, said differentiations comprising at least one of the following: early differentiation, before movement completion, and late differentiation based on later stages of the movement.

In some embodiments, the analyzer is adapted to analyze the motion patterns, and, upon identifying therein a primary sub-movement corresponding to an initial relatively large motion immediately followed by a secondary sub-movement corresponding to relatively small motion, classifying the motion pattern as corresponding to the goal directed movement.

The predetermined motion pattern segments may include successive segments indicative of, respectively, initiation of the goal-directed movement, undershoot and overshoot corrective sub-movements, and undershoot corrective sub-movement immediately before movement termination.

The selection and analysis of the motion pattern segment(s) is preferably performed by applying, to the goal-directed motion pattern, one or more machine learning models, which is/are trained on sensing data type(s) used in the monitoring system and various types of individual's activities. The learning and training process concerns identification of features of the motion profile over time in association with cognitive error detection by individual during such various activities.

The characteristic features include, for example, one or more of the following: submovements appearance in time relation to movement kinematics peak (maximum), submovements appearance in time relation to movement kinematics termination (undershoots, overshoots), temporal frequency of velocity derivatives changes, similarity of velocity derivatives changes across a time segment, temporal frequency of submovements; a time pattern of slopes of the kinematic change of the movement being terminated followed by the beginning of a successive movement, etc.

In some embodiments, machine learning based processing of the goal directed movement pattern associated with the certain individual's activity and being collected over time by the sensor of the predetermined type includes the following: sorting movements forming said motion pattern into correct movements and incorrect movements; identifying differences in features of the correct movements and the incorrect movements to define one or more characteristic feature uniquely characterizing errors; and determining a change in said one or more characteristic features resulting from a change in the individual's cognitive operational state, in association with each of one or more factors affecting the cognitive operational state of the individual.

In some other embodiments, the machine learning based processing to the goal directed movement pattern is association with the certain individual's activity and the sensor of the predetermined type is as follows: identifying in the motion pattern movements having different features; selecting one or more characteristic features from the movement located at extreme values of normally distributed motion pattern; and determining a change in said one or more characteristic features resulting from a change in the individual's cognitive operational state, in association with each of one or more factors affecting the cognitive operational state of the individual.

The control system may further include an operational state detector utility configured and operable to analyze the error-related motion pattern and generate operational state data characterizing the operational state of the individual.

In some embodiments, the error identifier utility is configured and operable to process the predetermined segments by analyzing motion command data of the individual's brain resulting in said motion pattern over corresponding reference motion command data, and determine error-related motion command data originated in the individual's brain.

In some embodiments, the error identifier utility is configured and operable to process the predetermined segments by analyzing motion command data of the individual's brain resulting in said motion pattern over corresponding reference motion command data, and determine error-related motion command data originated in the individual's brain; and the operational state detector utility is configured and operable to analyze error-related motion command data resulting in the error-related motion pattern over corresponding motion command error-related reference data.

The determination of the early differentiation may comprise analyzing the movement kinematics and determining a rate of movement kinematics development from a first measured motion command data value to movement kinematics maximum.

In some embodiments, the control system may be further adapted for recording data indicative of the cognitive error detection characterizing the cognitive operational state of the individual during said activity, thereby enabling use of the recorded data for optimizing corresponding error-related reference data.

In some embodiments, the control system is configured and operable to generate notification data indicative of the cognitive operational state of the individual during said activity.

The monitoring system may include a sensing system including one or more sensors (e.g., position sensor, gyroscope, accelerometer) configured and operable to provide the sensing data including the motion pattern measured over time during the individual's activity. The sensing system may include at least one accelerometer (e.g. piezoelectric accelerometer or semiconductor accelerometer material), and/or one or more force sensor (e.g. a force-sensing resistor or a piezoelectric sensor).

In some embodiments, the input data further comprises electroencephalography (EEG) data measured at the individual's brain, and/or electromyography (EMG) data measured at a skeletal muscle of the at least one body part of the individual.

According to another broad aspect of the invention, there is provided a monitoring system for monitoring an individual's activity, the monitoring system comprising a control system configured and operable to be responsive to input data comprising motion patterns measured over time by one or more sensors of predetermined one or more types and being indicative of motion characterizing the individual's activity, to process said input data and generate output data indicative of a cognitive error detection by said individual in said activity characterizing a cognitive operational state of the individual during said activity, wherein said processing of the input data comprises:
analyzing the motion patterns, and, upon identifying that the motion patterns comprise a pattern corresponding to a goal directed movement performed by the individual during said activity, generating corresponding decision data;
identifying one or more predetermined segments in the goal directed movement pattern including one or more segments from a first instant of the goal-directed movement until after a first instant of a latest motion pattern that served the decision of the goal-directed movement; and processing said predetermined segments,
analyzing motion command data of the individual's brain resulting in said motion pattern segments over corresponding reference motion command data, and determining error-related motion command data originated in the individual's brain; analyzing the error-related motion command data resulting in the error-related motion pattern over corresponding motion command error-related reference data; and
generating output data indicative of error-related motion pattern enabling evaluation of the operational state of the individual.

According to yet another aspect of the invention, there is provided an electronic device comprising: a sensing system including one or more sensors of one or more predetermined types, each configured and operable to provide sensing data including motion pattern measured over time during an individual's activity and being indicative of motion characterizing the individual's activity; and a control system configured and operable to be responsive to input data comprising said sensing data to process said input data and generate output data indicative of a cognitive error detection by said individual in said activity characterizing a cognitive operational state of the individual during said activity.

According to an additional aspect of the invention it provides a non-transitory program storage device readable by a computer, tangibly embodying a program of instructions executable by the computer to perform a method of detection of movements cognitively recognizable by an individual as error-related movements, the method comprising:
obtaining input data comprising motion patterns measured over time by one or more sensors of one or more predetermined types and being indicative of motion characterizing individual's activity;
analyzing the motion patterns, and, upon identifying that the motion patterns comprise a pattern corresponding to a goal directed movement performed by the individual during said activity, generating corresponding decision data;
identifying predetermined segments in the goal directed movement pattern, and processing said predetermined segments, and, upon determining that said predetermined segments are indicative of movements cognitively recognizable by the individual as error-related movements, generating output data indicative of error-related motion pattern enabling evaluation of the operational state of the individual.

According to yet further aspect of the invention, it provides a computer program product comprising a non-transitory computer useable medium having computer readable program code embodied therein for detection of movements cognitively recognizable by an individual as error-related movements, the computer program product comprising:
computer readable program code for causing the computer to obtain input data comprising motion patterns measured over time by one or more sensors of one or more predetermined types and being indicative of motion characterizing individual's activity;
computer readable program code for causing the computer to analyze the motion patterns, and, upon identifying that the motion patterns comprise a pattern corresponding to a goal directed movement performed by the individual during said activity, generating corresponding decision data; and
computer readable program code for causing the computer to identify predetermined segments in the goal directed movement pattern, and process said predetermined segments, and, upon determining that said predetermined segments are indicative of movements cognitively recognizable by the individual as error-related movements, generate output data indicative of error-related motion pattern indicative of the operational state of the individual.

The technique of the present invention can be used in various applications. For example, the functional utilities of the control unit may be installed in a personal communication device, e.g. via downloading them from a server system where the software product of the present invention is maintained. Such a communication device may be provided with motion sensors, e.g. accelerometer(s), and/or may be in signal communication with external sensor(s).

Generally, a steering wheel or any other type of vehicle control and maneuvering device or a smartwatch, a smartphone, an electronic wrist band or any other electronic device recording user's motor reaction or motor reactions derivatives can comprise or be operatively connected to the control system of the present invention, which may be configured to alert user's operational state or alert user's performance and/or initiate a command or a series of commands.

For example, a driver's operational state can be derived from the drive's error-detection patterns. These may include a ratio of movement duration, speed or length to the number of sub-movements or vice versa. It should, however, be noted that the exact error-detection pattern indicating the user's operational state can be set according to the characteristics of the movement of interest, be that for example a touch, a grip, a twisted or a straight movement, standing up, sitting down, continuous or a discrete movement, etc. This information can be used to acknowledge the driver or the car. For a semi-autonomous car this is of extreme importance as it helps the autonomous car to decide whether to accept a driver's request to take control, and later on, to deliver control back to the driver. Moreover, driver's error detection information can be correlated with specific levels of factors affecting the driver's operational state and used to alert when error-detection patterns reach a specific level indicating an illegal or a dangerous level for a specific factor.

In the case of measuring/evaluating a driver's cognitive state, the movement or position of the vehicle (the vehicle that is driven by said driver or other vehicle(s) in driver's environment) can be used as a measure in itself or as an addition to other measures. The reason for this is that when the driver controls the vehicle, the movement of the vehicle is a direct product of the decisions made by the driver and it is possible to identify in it the features that testify to a driver's cognitive state. In auto pilot mode, where the driver only partially controls the vehicle, a comparison between the motor activity of the driver in response to actual vehicle erroneous movements (the vehicle that carries the driver or other vehicles in driver's environment) and the actual vehicle movement can provide information about the cognitive condition of the driver. For example, if the driver responds motorically to braking or changes in the direction of movement of the vehicle or other vehicles, then the driver is cognitively competent.

In situations where information is available from only one source of information, while this might be insufficient and information from another different-type information source is needed, changes in the activity of the available information source can be indicative of changes in the missing type of information.

For example, while driving, the information is obtained only from steering wheel sensors. However, in order to get a complete picture, information from pedals is also required. In such a situation, the activity of the pedals can be detected/extracted from the information obtained from the steering wheel: when pedals are used, the body responds to changes in the vehicle's movement as a result of using the pedals and this reaction of the body is evident in the movement of the steering wheel.

For example, error-detection patterns can be matched against specific levels of blood alcohol levels. When driver's error detection patterns suggest that the driver has an illegal level of blood alcohol, an alert or command are issued. By the same token, error-detection patterns can be matched against specific levels of drowsiness. When driver's error detection patterns suggest that the driver reached a certain level of drowsiness, an alert or command are issued. Also, error-detection patterns can be matched against specific levels of attention. When driver's error detection patterns suggest that the driver reached a certain level of inattention, an alert or command are issued.

Also, it is well established in the scientific literature that the brain's error monitoring mechanism is also governing the body stabilization systems, and it was suggested that elders tend to fall because their error monitoring system is malfunctioning. Hence, error-detection signals evoke slightly before or after one is losing stabilization and about to fall.

Thus, an electronic device recording user's motor reaction or motor reactions derivatives such as a smartwatch, or an electronic band located on the wrist, the leg, around the waist and so forth, may serve to register a person's operational state related to a person's proficiency of stabilization mechanisms and issue an alert or command when a person's error detection patterns suggest that that person is at danger of losing stabilization and/or fall. Here, body stabilization motions can be recorded alongside or without movements of the limbs according to the methods described above.

Sometimes in order to properly monitor the individual's activity to extract data about a cognitive operational state of the individual during said activity, personal information regarding the individual is needed. This may, for example, include an individual's weight and/or gender. When the individual cannot be asked about this directly, this information can be obtained using independently operable sensor(s) (e.g. camera(s)) located in the surroundings of the individual. For example, a pressure or vibration sensor located under the driver's seat can provide information about the driver's weight.

Also, a person may be interested in receiving information about his/her own cognitive or physiological state. This is true for example for a person who is preparing for a meeting, or a person interested to learn what are his/her pick performance hours or days.

Also, an athlete or a surgeon interested in receiving information about his/her quality of movement planning and execution. For example, a tennis player may be interested in learning about his/her ability to initiate a perfect serve. Here, for example the number of corrective sub-movements and/or the timing of the corrective sub-movements relative to the beginning and/or end of the movement may be indicative as to how efficient was the serve.

Also, it is well established in the scientific literature that the brain's error monitoring mechanism rely on the same neural substrates deteriorating in Parkinson's disease. Hence, a person's error detection patterns can serve to indicate the severity of Parkinson disease symptoms. This can assist in determining appropriate medication dosages.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a block diagram exemplifying configuration of the monitoring system of the present invention;

FIG. 1B schematically illustrates the technique that can be used in the present invention for preparation of a machine learning model for use in processing measured motion patterns in association with sensors being used to collect such motion patterns and in association with individual's activity being monitored;

FIG. 2 is a flow diagram of the operation of the monitoring system of the invention;

FIG. 3 exemplifies the data processing technique of the present applied to an exemplary motion pattern measured/sensed at an individual in order to select segments of the motion pattern informative of whether the motion pattern corresponds to error-related motion related data or not, as a result of the operational state of the individual;

FIGS. 4A and 4B depicts experimental results showing how the driver's condition (sober or drunk) can be identified from his/her cognitive operational state using the technique of the invention;

FIGS. 5A and 5B depicts experimental results according to another example of using the technique of the invention to determine the driver's condition (fresh and tired) from his/her cognitive operational state;

FIGS. 6A-6B and 7A-7B show two examples, respectively, of the experimental results for determining the cognitive operational state of the individual from the motion patterns collected by steering wheel position sensors (FIGS. 6A and 7A) and under-seat sensors (FIGS. 6B and 7B);

FIG. 8 illustrates an example of measuring/sensing motion patterns by joystick sensors in a flight simulator;

FIG. 9 illustrates an example of measuring/sensing motion patterns by gaming console (joystick) sensors while a game is being played; and

FIG. 10 illustrates an example of measuring/sensing motion patterns from joystick sensors in a flight simulator.

### DETAILED DESCRIPTION OF EMBODIMENTS

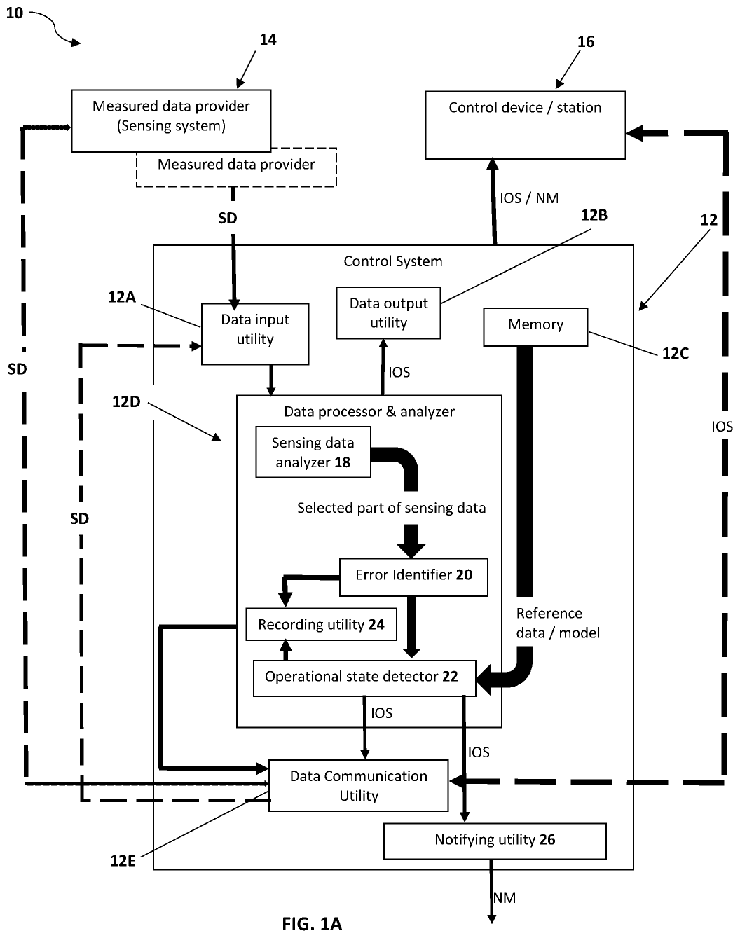

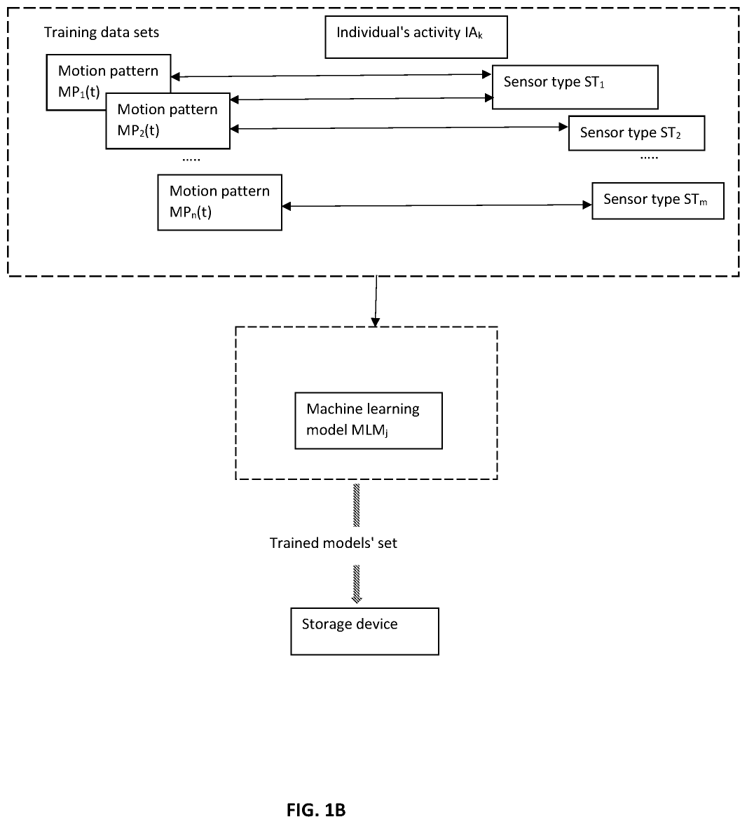

Referring to FIG. 1A, there is schematically illustrated, by way of a block diagram, functional parts of a monitoring system 10 of the present invention. The monitoring system 10 includes a control system 12 configured as a computerized system including inter alia data input and output utilities 12A and 12B, memory 12C, data processor and analyzer 12D, and a communication utility 12E of any known suitable type for signal/data communication (via wires or wireless communication of any known suitable type) with measured data provider(s) 14 and possibly also other control device(s) 16 (central control station), as the case may be. It should be understood that the measured data provider(s), as well as control device(s), may be integral with or external to (remotely connected to) the control system 12.

The terms “computerized device”, “computer”, “controller”, “processing unit”, “computer processor” or any variation thereof should be expansively construed to cover any kind of electronic device with data processing capabilities, such as a hardware processor (e.g. digital signal processor (DSP), microcontroller, field programmable circuit (ASIC), etc.) or a device which comprises or is operatively connected to one or more hardware processors including by way of non-limiting example, a personal computer, server, laptop computer, computing system, a communication device and/or any combination thereof.

The operations in accordance with the teachings herein may be performed by a computer specially constructed for the desired purposes or by a general purpose computer specially configured for the desired purpose by a computer program stored in a non-transitory computer readable storage medium. The presently disclosed subject matter further contemplates a machine-readable memory tangibly embodying a program of instructions executable by the machine for executing the disclosed method.

For the purposes of the present invention, the measured data provider 14 is associated with a sensing system, i.e. is the sensing system itself providing sensing data SD, or an external storage device where the sensing data SD, generated by the sensing system, is stored. As shown in the figure by dashed lines, the control system 12 of the present invention may be used with more than one measured data providers (data obtained from multiple sensing systems), e.g. to receive sensing data of different types.

It should be understood that, for most of applications, the data analysis is performed in real time, and thus the measured data/sensing data is provided by one or more sensors being in data communication with the system 10 and is analyzed in the online operational mode of the system 10.

Practically, in order to properly analyze and decide about the individual's cognitive operational state, the system utilizes data indicative of the individual's current activity to be monitored. Such activity-related data may be entered into the system as part of the input data and/or may be identified by the system from the sensing data itself. For example, the individual activates the system (or a relating operational device, e.g. a sensor itself) by himself/herself and the individual's activity is thus properly identified by the system.

Thus, the data utilized by the data processor and analyzer 12D includes the individual's activity related data and the type of the motion being sensed in relation to that activity. In some applications (e.g. monitoring the individual's activity in a vehicle, e.g. while driving the vehicle and/or while controlling/monitoring an autonomous or semi-autonomous vehicle) various types of sensors can be concurrently involved. The data input utility may for example properly tag the sensing data received from a certain sensor with the sensor-related data (identification data).

Preferably, the data processor and analyzer 12D processes the motion pattern by applying thereto at least one machine learning model being previously properly trained using training data set (motion pattern(s)) measured by given one or more sensors (i.e. one or more sensors of one or more predetermined types) with respect to a given type of individual's activity. For example, the individual's activity may be monitored by two or more different sensors, e.g. associated with motions of different parts of the individual's body. In this case, either different models are trained on different training data sets, respectively, and the resulting trained model describes a combined set of features, or a hybrid model is trained using two or more inputs of the two or more training data sets.

This is exemplified in a self-explanatory manner in FIG. 1B. As shown, training data set is prepared including N motion patterns MP₁ . . . MPₙ (generally, N≥1) measured by sensors of M types ST₁ . . . STₘ (generally, M≥1), in association with a given k-th individual's activity IAₖ. At least one selected j-th machine learning model MLMⱼ is trained on this data set. The so-obtained trained model is stored in a storage device accessible by the data processor and analyzer 12D. It should be understood that the same sensor-type may be used for measuring two or more motion patterns from different parts of the individual's body, as for example shown in the figure with respect to the motion patterns MP₁(t) and MP₂(t) being collected by the sensor type ST₁.

The machine learning model to be trained for the purposes of the present invention may be of any known suitable type. For example one or more of the machine learning models known as Lasso, multi task elastic net, Bayesian Regression, Logistic regression, Ridge, SVM can be used.

The learning and training process concerns identification of features of the motion profile over time in association with cognitive error detection during various individual activities. The characteristic features include, for example, one or more of the following: submovements appearance in time relation to movement kinematics peak (maximum), submovements appearance in time relation to movement kinematics termination (undershoots, overshoots), the ratio of movement's ascending slope (or part of movement's ascending slope) to movement's descending slope (or part of movement's descending slope), the ratio of different parts of movement's ascending slope, the ratio of different parts of movement's descending slope, temporal frequency of velocity derivatives changes, similarity of velocity derivatives changes across a time segment, temporal frequency of submovements; a time pattern of slopes of the kinematic change of the movement being terminated followed by the beginning of a successive movement, etc.

In order to label the relevant features for the purpose of identifying individual's cognitive operational state two methods may be used.

In the first method, the measured responses (motion patterns) in a given context associated with a given individual's activity (e.g. driving a vehicle, flight activity, a particular sporting activity, smartphone use) collected from a given sensor (e.g., steering wheel position, under thigh, smartwatch, smartphone) are first sorted into correct movements and incorrect movements. To this end, user's input may be considered, as well as other information source, such as simulator or task output (collision). A machine learning model then identifies the differences in the expressions of different features between the correct movements and incorrect movements. In the next step, the feature expressions found to be uniquely characterizing errors are fed to a machine learning model that studies how these features expressions change as a result of changes in a person's cognitive operational state. This test is performed separately for each factor that affects the cognitive operational state (e.g. different levels of alcohol in the blood, different levels of fatigue, different levels of fatigue, different levels of attention).

The second method relies on the fact that while analyzing the data from the first method, the inventors found that the main characteristic that distinguishes incorrect movements from correct movements is that the expression of the different features in incorrect movements is in most cases located at the extreme values of a normally distributed data. That is, if correct responses and incorrect responses are analyzed together, without prior sorting, the expression of many features within an incorrect movement is found at the extremes of the normal distribution. For example, variation of the signal's spectrum in extreme movements (the 85 percentile), the extreme values of the angle of the Fourier Transform (the 95 percentile of the values), the entropy of the power spectral density (lower 25 percentiles of the values), the extreme values of the p-value indicating that the linear trend of the move is zero (the extreme values i.e. the 98 percentile of the values), mass center of the signal, share of positive values in a movement—(the extreme values i.e. the 95 percentile of the values). Therefore, in the second method sorting or pre-distinction between correct and incorrect responses before analysis may not be performed. The machine learning model looks for different features within a set of responses from a particular context, collected from a given sensor over a period of time and then uses only movements where the expression of the features is at the extreme values of the normal distribution. It then examines how these features are affected by a change of a person's cognitive operational state. This test is performed separately for each factor that changes the cognitive operational state (e.g. different levels of alcohol in the blood, different levels of fatigue, different levels of fatigue, different levels of attention).

As described above, the control system 12 and the sensing system 14 including one or more motion sensors may be integral within a handheld electronic device, such as an individual's personal communication device. For example, an electronic device, such as individual's personal communication device, typically including one or more motion sensors as well as data presentation utilities (in audio and/or visual data presentation format), may be configured for downloading software utilities of the control system (the operation of which will be described more specifically further below) from a server system where a corresponding software product of the present invention is maintained. Such electronic device (e.g. individual's personal communication device) is thus configured and operable as the monitoring system of the present invention.

The sensing system includes one or more sensors adapted to provide sensing data SD to be processed and analyzed by the control system 12. The sensing data SD includes motion data MD indicative of movements or kinematics of at least one relevant part of individual's body being sensed during a sensing time. In other words, the motion data MD includes a number N (N≥1) motion patterns, each i-th motion pattern (i=1 . . . n) measured over time, MPᵢ(t).

It should be understood that such movements/kinematics may be directly measured/sensed at the at least one body part, and/or derived from data measured at a certain device being operated by the individual. For example, the measured motion pattern may be indicative of force related data (a force or derivative thereof) corresponding to a force applied on the device and/or may be indicative of a time to lift of the at least one body part from the device, and/or indicative of an eye movement, and/or voice command, and/or facial muscles movement.

In another non-limiting example, the measurement of error-related motor activity in the muscles of the body may be performed, e.g. in the muscles of the leg or thigh or back (e.g., when a person is driving a vehicle). The sensor or sensors can be placed on the driver's body and/or at the driver's seat (e.g., under the thigh or at the back of the seat). Alternatively or additionally, a camera (imaging sensor) can be used.

Such activity can indicate the cognitive operational state of the driver in a situation where the driver is holding the steering wheel and operating the car, and also in a situation where the driver is not holding the steering wheel and only watching the car's autopilot operation. The driver body's motor activity responds to both the driver's decision and the autopilot's decisions (whose operation might be watched by the driver) and the driver's operational state can be determined from such responses, for example, it can be determined whether the driver monitors the vehicle's activity or the autopilot's decisions.

The monitoring procedure can be implemented as a closed loop process. More specifically, the system can identify, from the characteristics in motor activity, the decisions of the driver or certain actions of the autopilot without receiving information from the vehicle (vehicle's controller).

Alternatively, the monitoring procedure can operate on the basis of information received from the vehicle's controller. For example, when the vehicle's activity is indicative of that it operates to correct a steering error or brakes, the monitoring system checks whether there is a reaction of the driver's motor activity to error correction made by the vehicle. Examples of the driver's motor activity response may include a corrective sub-movement and/or the driver's activity that reflects one or more of the following: a tilt in the direction of the vehicle movement or in the opposite direction; a sudden or gradual increase in muscle tension or muscle tremor; a level/degree of synchronization between the actions of the vehicle or autopilot and the actions of the muscular system of the driver;

a degree of synchronization between different muscle systems. The latter may include: synchronization between hand movement and eye movement, or between head movement and body movement, or between back movement and hip movement.

In some embodiments, the sensing data SD may include, additionally to the motion data, electroencephalography (EEG) data measured at the individual's brain, and/or electromyography (EMG) data measured at a skeletal muscle of the at least one body part, and/or autonomic nervous system reaction data (including but not limited to the following parameters: cardiovascular, electrodermal, respiratory, which may be reflected but not limited to changes in heart rate, heart rate variability, blood pressure, blood pressure variability, blood flow, efferent postganglionic muscle sympathetic nerve activity (microneurography), skin electrical conductance or temperature, pupillary response (including differences between pupillary responses of the right and left eyes), eye blood vessels response and its derivatives, muscle tone etc.).

The measured/sensed motion pattern or multiple motion patterns describe(s) certain activity or activities of the individual and characterizes an operational state of the individual during said certain activity. Such motion pattern or patterns may include movements indicative of motion command data characterizing the reaction of the individual to his/her own errors, which is to be identified by the system of the invention to determine the operational state of the individual (i.e., cognitive, emotional, physiological etc.) and/or quality of motor functioning.

Thus, the control system 12 receives input data (from the measured data provider 14) indicative of the sensing data SD including at least the motion pattern(s) measured over time, MP(t). This sensing data SD may be directly received from the sensing system 14, or via wireless communication utility 12E (using any known suitable wireless communication techniques and protocols). The sensing data SD is analyzed by the data processor and analyzer utility 12D, which generates resulting output data indicative of the individual's operational state IOS.

The control system 12 may include a notifying utility 26, which receives the data indicative of the individual's operational state IOS and generates a corresponding notification message NM (in any suitable format) to be presented by the control system to the individual and/or to authorized person/entity and/or to a machine reacting to or operated by the user. Alternatively, or additionally, the data indicative of the individual's operational state IOS and/or the corresponding notification message may be transmitted to the control device 16 (e.g. for further analysis and monitoring). As will be described further below, the control system may include a recording utility 24 for recording the data indicative of the individual's operational state IOS and/or data indicative of detected error-related motion command data (determined as described below), to be used for further determination/optimization of motion command data statistics.

The data processor and analyzer utility 12D includes a sensing data analyzer 18; error identifier 20; and operational state detector 22. The optionally provided recording utility 24 may or may not be part of the data processor and analyzer 12D.

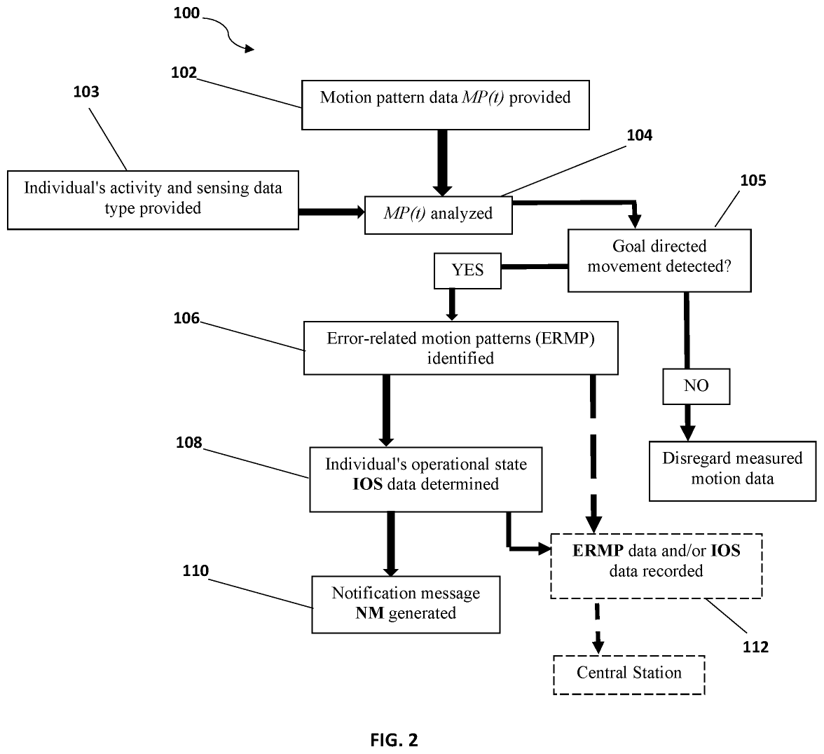

The operation of the control system 20 is exemplified by a flow diagram 100 of FIG. 2. Sensing data SD, indicative of the motion data (one or more motion patterns) sensed over time MP(t), is provided and received by the data analyzer 18 (step 102). As described above, also provided to/identified by the control system is the sensing type data and data indicative of the individual's activity to be monitored (step 103).

The data analyzer 18 is configured and operable to analyze the received motion MP(t) (step 104), and upon determining/deciding that the motion being sensed is of a goal directed type (step 105), allow further processing of the motion data by the error identifier 20.

The goal directed motion pattern can be identified using early differentiation (before movement completion), and late differentiation based on later stages of the movement. The determination of the early differentiation may include analysis of the movement kinematics to determine a rate of movement kinematics development from a first measured motion command data value to movement kinematics maximum.

In some embodiments, the determination of the early differentiations may include the following: The movement kinematics starting from a first measured motion command data value originated in the individual's brain at a first instant along the motion pattern of executed command is analyzed. In some instances, the rate of movement kinematics development from the first measured motion command data value to movement kinematics maximum is sufficient to determine a goal directed movement. In other instances, based on the first measured motion command data value, a motion command data value is expected/predicted for a later instant along the executed command or action, and compared with the measured motion command data value at the later instant. Upon identifying that a relation (e.g. difference) between the expected motion command data value and the measured motion command data value complies with a predefined criterion, the motion pattern is classified as corresponding to the goal directed movement.

It should be noted that the term “criterion” as used herein should be expansively construed to include any compound criterion, including, for example, several criteria and/or their logical combinations.

A late differentiation can be based on indications at later stages of a movement. For example, any indication that a goal has been at least partially achieved, renders the movement a goal directed movement.

In some embodiments, the data analyzer 18 is adapted to analyze the motion patterns, and, upon identifying therein a primary sub-movement corresponding to an initial relatively large motion immediately followed by a secondary sub-movement corresponding to relatively small motion, classifies the motion pattern as corresponding to the goal directed movement.

It should be understood that various types of individual's activity are by definition associated with goal directed maneuvers. These are for example human-machine interactions. When a user is operating an electronic device, such indication may be received from the electronic device.

When a user does not operate an electronic device but rather initiates an action aimed at completing a certain task or reaching a certain goal (i.e., shoe tying, grasping a glass of water, standing up) or operates a non-electronic device (i.e., tennis racket), indication may be received from the user's own movements.

The inventors have found that kinematic pattern, and possibly also EMG parameters, indicative of the movement itself may provide information that the movement ended up with a goal (e.g., a touch or a grip). For example, kinematic patterns (motion patterns) accompanying a touch are reflected by sudden halt of movement velocity with rather long period before movement velocity is regenerated. EMG patterns accompanying a touch are reflected for example by sudden and/or early halt of EMG bursts with rather long period before EMG bursts are regenerated. Kinematic patterns accompanying a grip are reflected for example by sudden halt of movement velocity immediately followed by short acceleration bursts. EMG patterns accompanying a grip are reflected for example by sudden and/or early halt of strong EMG bursts followed by small rapid EMG bursts.

The ability to identify goal directed movements (e.g. touch or grip) can be facilitated by using a sensing system including an accelerometer, preferably of a gyroscope type. This is because a gyroscope measures or maintains orientation and angular velocity. A gyroscope can advantageously be used for identification goal directed whole body motions, such as standing up, sitting down, turning and so forth; as well as for identification of goal directed motions where one is eating because both grasping a fork and reaching the mouth involves orientation and angular velocity changes.

Additional indication of a goal directed movement is an initial large motion (i.e., primary sub-movement) immediately followed by small motions (i.e., secondary sub-movement) which indicates that a purposeful movement was initiated, however it wasn't accurate enough so it was immediately corrected.

Yet further indications may be the relations between kinematic patterns or EMG parameters of the movement in question and kinematic patterns or EMG parameters of the recovery phase of that movement.

The error identifier 20 is configured and operable for applying a segmentation processing to the motion pattern data to identify at least one part/segment and analyze said selected segment(s) to identify whether it is indicative of error-related motion patterns ERMP (step 106).

The selected motion pattern segments may include successive segments indicative of, respectively, initiation of the goal-directed movement, undershoot and overshoot corrective sub-movements, and undershoot corrective sub-movement immediately before movement termination.

The error identifier 20 searches for the predetermined segments in the goal directed motion pattern which include motion from the first instant of the goal-directed movement until after the first instant of the latest motion pattern that served the decision of goal-directed movement by analyzer 18. These selected segments of the motion pattern may be indicative of the movements executed by the individual, while being affected by error-related motion command data originated in the individual's brain in response to the individual's cognitive recognition of his/her own error in the performance during certain activity, and thus correspond to the error-related motion patterns ERMP (at times referred to as “error related detection patterns”).

The analysis may be based on predetermined threshold values to determine whether the comparison complies with one or more predefined criteria indicating that a given measured motion command data/value corresponds to an error cognition cognitively recognized by the individual as an erroneous action. The analyzer 18 may use any known suitable type of feature-based data analysis for the comparison procedure.

Some specific but not limiting examples of features include the following classic features: mean, standard deviation (STD), median, maximum (max), minimum (min), a relation (e.g. difference) between maximum and minimum, the signal magnitude area (SMA), skewness, kurtosis, zero crossing (time over zero), mean frequency, median frequency; sample entropy and LempelZiv complexity.

Preferably, as described above, the error identifier 20 is preprogrammed with one or more trained machine learning models each dedicated for motion pattern analysis in association with the sensing type data and the specific activity of an individual. More specifically, the motion pattern being sensed over time is divided into time slots/segments and they are successively analyzed to properly identify the first appearance of the segment containing features characterizing error-related motion pattern ERMP. Some examples of features characterizing error-related motion patterns ERMP in various types of individual activity will be described further below.

In many cases effective differentiation between goal directed and non-goal directed movements is based on a combination of both the early parameters and the late parameters. This also allows accurate segmentation of the movement of interest, i.e. search for and analyses of the predetermined parts/segments of the received motion pattern to determine the error detection related patterns ERMP which present corrective sub-movements. This is for example because in some usages the number of error detection related patterns ERMP (e.g., corrective sub-movements) within a movement segmentation (or across several segmentations) serves to indicate the quality of user's state or performance. It should, however, be noted that error detection related patterns ERMP (e.g., corrective sub-movements) occurring slightly after the first indication of movement segmentation conclusion, may in fact be related to the preceding movement and hence clustered with error detection related patterns occurring across the most recent segmentation or segmentations. The segmentation process will be described more specifically further below.

It should be noted that data analyzer 18 may further be configured to process the raw sensing data received from the sensing system in order to extract desired motion data and/or to transform the data to a desired format (performing operations such as noise filtering, artifact rejection, etc.). The desired motion data may be extracted, for example, by identifying the frequency range of the specific movement that is chosen for analysis and filtering information that is not in the relevant frequency range. This effective filtering method is more sophisticated than the classic noise filtering method which usually filters extremely low or high frequency ranges which are commonly identified with noise.

The method used in the invention is unique because the frequency range of the signal that being of interest for the data analysis is first detected, and this frequency range depends on the sensor used for measurement. This range can be different for different types of measurements, such as: measuring the motion of a steering wheel by measuring the change in angle of the steering wheel, measuring the EMG of the muscle that activates the steering wheel, measurement of a pressure sensor or accelerometer or gyro, etc.

The error-related motion command data corresponding to the error-related motion patterns ERMP is analyzed by the operational state detector 22 in relation to corresponding error-related motion command reference data (i.e. error-related reference motion patterns occurring in a similar movement, e.g. similar limb, kinematics, force, distance traveled, direction, movement goal, error size, context etc.), to determine the individual's operational state IOS data (step 108). Based on this data analysis, the notifying utility 26 may generate notification NM including data/message regarding the individual's operational state, and/or warning pertaining to the user's state and/or quality of motor functioning (e.g. by displaying a dialog box on a display device connected to the control system (step 110).

As also shown in the figure, data indicative of the error-related motion patterns ERMP and/or data indicative of operational state IOS may be duly recorded (step 112) and may be communicated to a central control station.

Thus, the analyzer 18 identifies and selects the goal directed motion patterns to be processed by the error identifier module 20. The latter operates to determine whether the motion pattern corresponds to movement progress deviation from the initial movement plan or goal, where such deviations may occur during well-defined discrete events or during continuous and not easily parsed motions; and identify and analyze different types of deviations indicative of error detection in the individual's brain.

Such movement progress deviation from the initial movement plan or goal may include but are not limited to, sub-movements in the motion pattern, as described above. The data processing technique includes comparison of the measured error detection related deviations with pre-stored or reference error detection related deviations, in order to determine whether measured error related deviation, or the difference value between measured and reference error related deviations, indicates a change in the individual's cognitive operational state.

According to one example, differences can be detected between measured motion command values/data characterizing the reaction of an individual to his/her own errors, and predetermined motion command values characterizing the reaction of the same individual or other individuals to proper operation or action (reference data). The individual has certain expectations as to how his/her own operation or action is supposed to proceed. The measured values characterizing the reactions of the individual to own actual operation or action can be indicative of whether the expectations are met or not. The response of the individual to an unexpected deviation from original operation or action plan, or to a need to update the original operation or action plan, is sensed via the analysis of the motion patterns and is considered an incorrect action, as compared to an individual's response when expectations are met or when there is no need for update.

In other words, based on the measured data, it can be determined whether the motion related command originated in the individual's brain is an erroneous command (e.g. motion command driven by an incorrect decision) or a non-erroneous command (e.g. correct, driven by a correct decision). In the present application, the term “error related motion pattern” or “error related detection pattern” is used as a general term to include any one of: incorrect decision, erroneous command and erroneous action.

If it is determined, from the machine learning based analysis of the sensed motion pattern characterizing the reactions of the individual to his/her own decision, motion command or actual operation or action, that the decision, motion command or actual operation or action deviates from expected one or needs to be changed, this information is then analyzed by the operational state detector 22, and can be used in various ways.

For example the individual can be informed, via a respective notification message NM, as to how efficient was his/her own response to own error. For example, the individual may receive notification message NM including information regarding the difference between the measured data indicating an error and corresponding reference data indicating a correct action. A large difference may indicate an efficient response to errors. Generally, in response to detection of error related detection/motion pattern ERMP, various preventative actions can be carried out in order to abort, correct or otherwise react to such detection, as well as record the corresponding operational state of the individual.

In another example operational state detector 22 may use information from error-related motion patterns ERMPs collected over time to inform, via a respective notification message NM, as to how efficient was his/her own average response to own errors. For example, the individual may receive notification message NM including information regarding the difference between the measured data (e.g. average measured data) indicating an error and corresponding reference data (e.g. average reference data) indicating a correct action or an error. A large difference may indicate an efficient or an inefficient response to errors, depending on sign (positive vs. negative) of difference. In another example, the individual may receive the notification message NM including information regarding the probability that an upcoming decision, motion command or actual operation or action will deviate from an expected one (e.g., an upcoming error, risky behavior, etc.). Generally, in response to detection of error related detection/motion pattern ERMP, various preventative actions can be carried out in order to abort, correct or otherwise react to such detection, as well as record the corresponding operational state of the individual.

As described above, the operational state of the individual being monitored/controlled may be associated with individual's operation of a device/machine which may be of any suitable type including, but not limited to, any one of the following devices: smartphone, smartwatch, computer keyboard, computer mouse, touch-screen, touch-pad, mechanical or electronic lever, mechanical or electronic button, mechanical or electronic switch, mechanical or electronic knob, mechanical or electronic trigger, mechanical or electronic paddle, gesture based touch-less computer interface operated by any type of body part (e.g. based on a camera and a computer screen), eye movement computer user-interface, voice command computer user-interface, etc.

Also, as described above, the individual's activity being monitored/controlled may include activities which do not necessarily have a direct effect on the operation of any device/machine (e.g. when the individual's activity includes only his/her observation of the device operation or a smartwatch or any wearable device recording biological reactions of an individual) or activity where the individual does not deliberately operates any device.

For example, the case may be such that an operator is observing the operation of a device and the only interaction with the device is through the observation. The operator's cognitive command data affecting the reactions of the operator to the observed operation of the device can be monitored and used for detecting operator's state. Also, the cognitive command data affecting the reaction of an individual to own actions can be monitored and used for detecting errors.

The motion command data being measured/sensed may include kinematics measured in relation to the individual's body part involved in the individual's activity. Kinematics include for example velocity of the body part during said activity, acceleration of the body part during said activity, deceleration during said activity, etc. The kinematics measured when erroneous action is performed are different to those measured when a non-erroneous (e.g. correct) action is performed.

Alternatively or additionally, the motion command data being measured/sensed may include kinematics measured in relation to the individual's operation of a certain device, responsive to the respective body part. Such kinematics includes for example velocity, acceleration or deceleration of the device when responding to individual' action. The kinematics measured when erroneous action is performed are different to those measured when an action resulting from a non-erroneous command is performed.

Kinematic measures may include corrective sub-movements. Purposeful movements often require high degree of accuracy. In order to achieve accuracy, efficient control is needed, mostly to avoid an increase in spatial errors. Purposeful movements often include small discrete phases or irregularities or sub-movements. A purposeful movement is composed of a series of ballistic sub-movements (e.g., undershooting and overshooting). Undershooting is the primary sub-movement that falls short of the target, then the secondary movement hits the target. Accordingly, overshooting is the primary sub-movement that overshoots the target, then the reverse movement (secondary sub-movement) hits the target.

Sub-movements could be identified in the measurement of pressure or force. Even when applying a pressure or force, there are slight corrections for pressure or force that is too weak or too strong while applying the pressure or force.

Subsequent sub-movements usually result from visual information and other feedback obtained from the variability of a current or previous sub-movement. In the absence of vision or in addition to vision, the corrective process is based on proprioceptive or kinesthetic information.

Because sometime sub-movements are too fast or too early to be based on feedback processing, these sub-movements may be based on feedforward processes determined from a motor program before a movement begins. Some models are based on assumption that execution of the initial impulse or primary sub-movement is affected by neural noise in the motor system. In this case, corrective sub-movements are assumed to be only occurring once the primary sub-movement is anticipated to miss the target. Accordingly, sub-movements in the final portion of a discrete movement are viewed as movement corrections. However, continuous control models are based on that initial adjustment might not be ballistic and that corrective sub-movements may occur along the whole course of a movement. In these models, differentiating non-corrective from corrective sub-movements is a challenge. It should, however, be noted that sub-movements associated with acceleration profile deviations related with gradual reduction of braking force of active limb toward the target can be differentiated from corrective sub-movement by the lack of velocity increase. Even the notion that secondary movements are corrective in nature had been challenged. Many of the sub-movements are considered to arise from biomechanical sources of movement variability and may not be corrective fluctuations.

Generally, there are three types of sub-movements including: sub-movements which are zero crossings from positive to negative value occurring in a single velocity profile (type 1); sub-movements which are zero crossings from negative to positive value occurring in the acceleration profile (type 2); and sub-movements which are zero crossing from positive to negative value occurring in the profile of derivative of acceleration/deceleration in relation to time (type 3). The type 1 sub-movements may reflect overshooting; the type 2 sub-movements may reflect undershooting, and pre and post peak sub-movement may reflect type 3. The type 1 sub-movements often emerge due to motion termination; type 2 sub-movements are associated with either motion termination or accuracy regulation; and type 3 sub-movements relate to motion fluctuations when movement speed decreases.

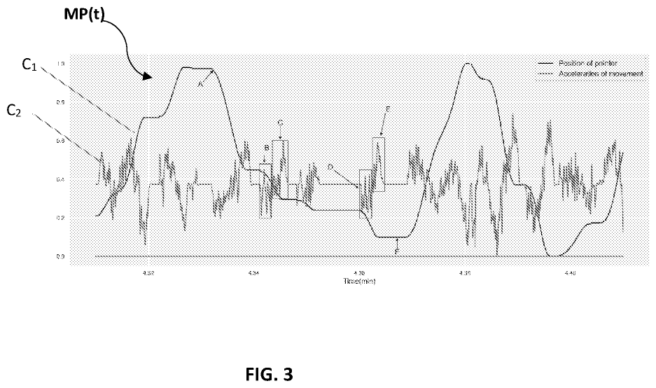

The segmentation technique used in the error-detection data analyses of the motion pattern, based on the differentiation of the sub-movements, is exemplified in FIG. 3. In this figure, the measured/sensed motion pattern MP(t) is shown represented by a curve C₁ corresponding to direction changes of a continuous hand motion and curve C₂ representing motion acceleration changes. Segment A of the curve C₁ presents initiation of goal-directed movement; segment B presents undershoot corrective sub-movement immediately after pick acceleration; segment C corresponds to overshoot corrective sub-movement immediately after pick acceleration; segment D presents undershoot corrective sub-movement immediately before movement termination; segment E presents overshoot corrective sub-movement immediately before movement termination; and segment F presents termination of the goal-directed movement.

The characteristics and prevalence of sub-movements can result from different task constraints. As aforesaid, in the context of the current application, the sub-movements are to be interpreted as corrective adjustments because such sub-movements reflect a specific case of error detection in an individual's brain.

However, there are other sub-movements interpretations. For example, sub-movements may be interpreted as a property of movement control. More specifically, sub-movements may be movement primitives used as building blocks of normal movements, thus having no direct relation to accuracy requirements. Also, many sub-movements may represent irregular velocity fluctuations, emerging due to noise in the kinematic output (i.e., muscle elasticity, co-activation, non-smooth activation of motor units, and noise in the neural circuitry involved in movement control).

Thus, it might be difficult to distinguish corrective and non-corrective sub-movements based on kinematic analyses. Corrective and non-corrective sub-movements have similar kinematic characters, reflected by velocity profile modulations, usually measured by zero crossings of the first three or four displacement derivatives. The distinction may be based on fitting movement trajectory with series of bell-shaped functions of scaled duration and amplitude. However, sub-movements extracted according to these methodologies can be either corrective or non-corrective. According to some other approaches, if the sub-movement brings the trajectory closer to the target it is a corrective one. However, noisy target-aimed motions may have the same or similar characteristics as a series of corrective sub-movements. According to yet further approaches, motion termination may cause sub-movements because it requires dissipation of movement mechanical energy and stabilization of the arm at the target. In discrete movements, motion termination results in complete halt of both velocity and acceleration. However, in continuous movements that reverse without residing on target, only the velocity, and not acceleration, is abolished at the target. The stabilization of the limb at the target in discrete motions may cause sub-movements, absent in continuous movements. Accordingly, sub-movements revealed with the lower derivatives (gross sub-movements) are often caused by motion termination in discrete motions but not in continuous motions. Conversely, sub-movements revealed with higher derivatives of motion (fine sub-movements) might be more related with corrective maneuvers associated with higher accuracy demands and occur in both discrete and continuous motions. However, during cyclical movements, incidence of fine sub-movements depends on cyclic frequency (frequency of periodic movement) and not on accuracy demands. Hence, slow movements may be prone to irregularities observed as fine sub-movements, and since highly accurate motions are also slower, these movements are characterized by non-corrective fine sub-movements.

Alternatively or additionally, the motion command data being measured/sensed may include a force (or any derivative thereof such as pressure) applied by the body part (e.g. on a certain device) when performing the action. In general, the force applied when erroneous action is performed is different from the force applied when a non-erroneous action is performed. The applied force can be measured on the individual's body part which is applying the force or on the device on which the force is being applied. Similar to the acceleration and the derivative of acceleration/deceleration in relation to time, the rate of change in the applied force can be calculated and used as an indication of an erroneous action.

The motion command data being measured/sensed may also include time to lift parameter being measured as a time interval before the body part is lifted from a certain device on which the action is applied, or a time interval before the pressure applied on the device is alleviated, or a time interval before an electric circle closed by the action, opens again. For example, a time to lift period can be measured from the moment of initial contact of a body part with the device until the body part is lifted from the device. In general lifting time shortens when the action is a result of an erroneous command as compared to an action which results from a non-erroneous command.

The sampling frequency used in measurements of error detection-related kinematics and other information related to the measurement of motion command data is preferably above 50 Hz and, preferably, above 100 Hz.

The following are some examples of simulations and experiments conducted by the inventors demonstrating how the technique of the present invention can be used in monitoring various individual's activities.

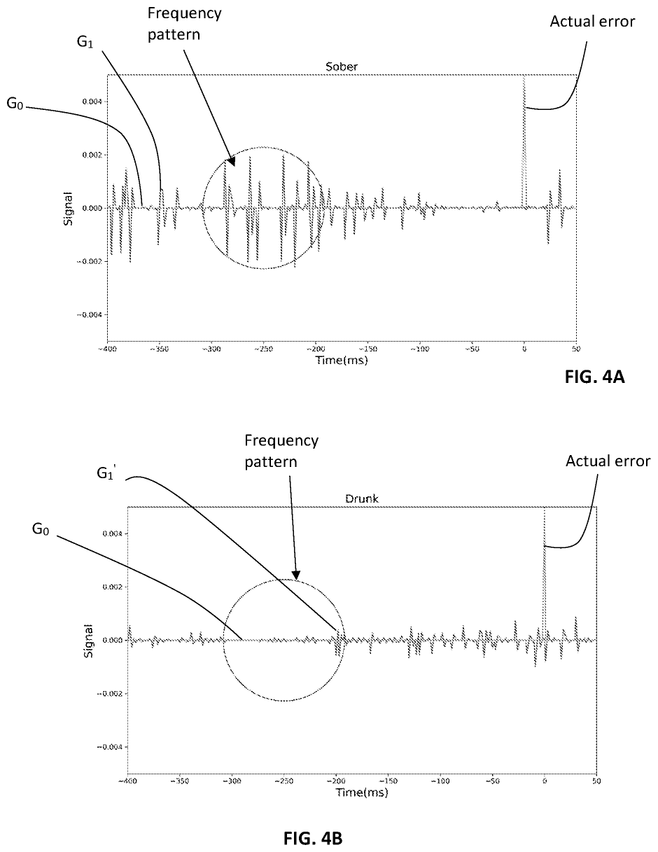

FIGS. 4A and 4B illustrate experimental results showing how the driver's condition can be identified from his/her cognitive operational state using the technique of the invention. In this example, the cognitive operational state of the driver is determined by analyzing sensing data describing a change of the angular position of the steering wheel with time as a result of its operation by the driver, i.e. this time change presents the motion pattern affected by the cognitive operational state of the driver during the car driving. FIG. 4A shows the cognitive operational state corresponding to the normal condition (sober), and FIG. 4B shows the cognitive operational state corresponding to the abnormal condition (drunk). The figures present time functions G₁ and G₁′ of the variation of the angular position of the steering wheel expressed by the acceleration measure (or any other function describing the velocity of change of such position). The figures show the number of velocity derivatives (here, accelerations) within a fixed period of time. Here, curve G₀ presents the error state evolution, where the peak corresponds to the actual error occurrence (collision). As can be seen the feature characterizing the cognitive operative state of the driver via error corrective movements (or submovements) is expressed by a temporal profile (frequency pattern) of the change in the motion pattern.

The abnormal condition (FIG. 4B) is characterized by significantly lower density and amplitude of the error corrective movements. It basically shows that the more competent a person is (FIG. 4A), the stronger is that person's error related activity. The derivatives indicate that the brain made repeated attempts to cancel or correct the error, “fighting” with the more potent deliberate erroneous movement.

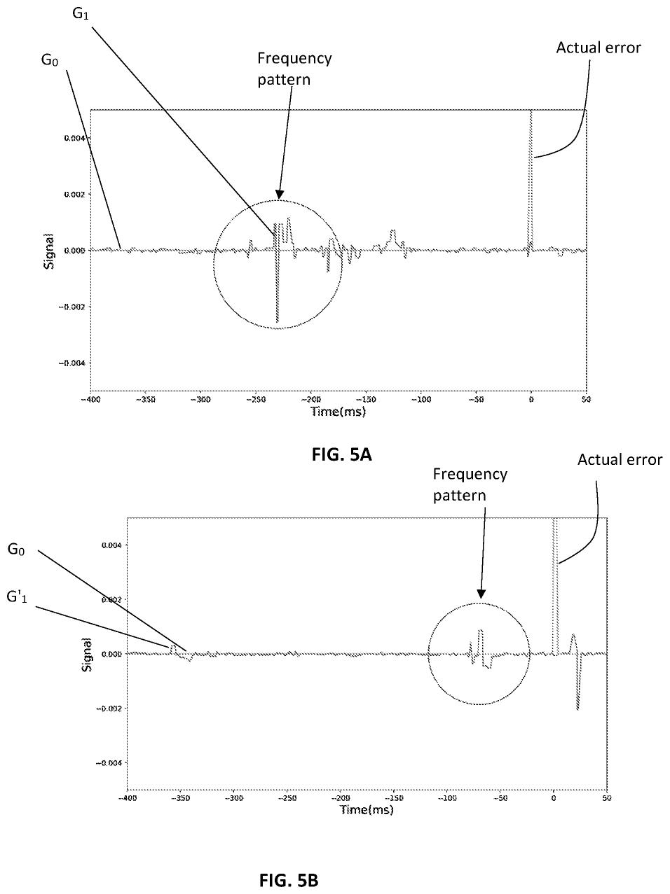

FIGS. 5A and 5B illustrate experimental results exemplifying the use of the invention to determine the driver's condition (fresh and tired) from his/her cognitive operational state. Similar to Figs, 4A and 4B, here time functions G₁ and G₁′ correspond to the variation of the angular position of the steering wheel expressed by the acceleration measure (or any other function describing the velocity of change of such position), and curve G₀ presents the error state evolution, where the peak corresponds to the actual error occurrence (collision). As can be seen, the feature characterizing the cognitive operative state of the driver via error corrective movements (or submovements) is expressed by a temporal profile (frequency pattern) of the change in the motion pattern and the time slot of the appearance of such profile in relation to actual error occurrence

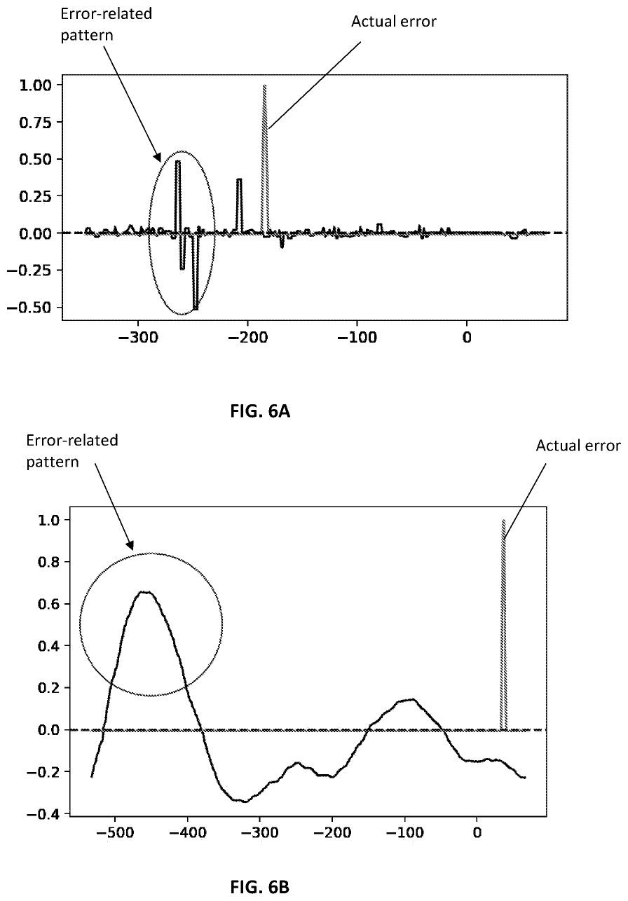

FIGS. 6A-6B show experimental results for determining the cognitive operational state of the individual from the motion pattern collected by steering wheel position sensor (FIG. 6A) and under-seat sensor (FIG. 6B). FIG. 6A shows the error-related motion pattern measured/sensed from the steering wheel position sensors just before a driving error (driver accelerates, failing to notice a curve), and FIG. 6B shows error-related motion pattern measured/sensed from the under-seat sensors just before a driving error (driver gets too close to a neighboring car).

FIGS. 7A and 7B show another example of the error-related motion patterns measured/sensed from steering wheel position sensors (FIG. 7A) and from under-seat sensors (FIG. 7B). Curves C₁ and C′₁ depict the movements; and curves C₂ and C′₂ depict, respectively, the acceleration analysis conducted to extract (from the steering wheel motion pattern) an overshoot sub movement appearing around movement termination, and the acceleration analysis conducted to extract (from the under-seat sensed motion pattern) sub movements appearing immediately after movement peak (maximum).

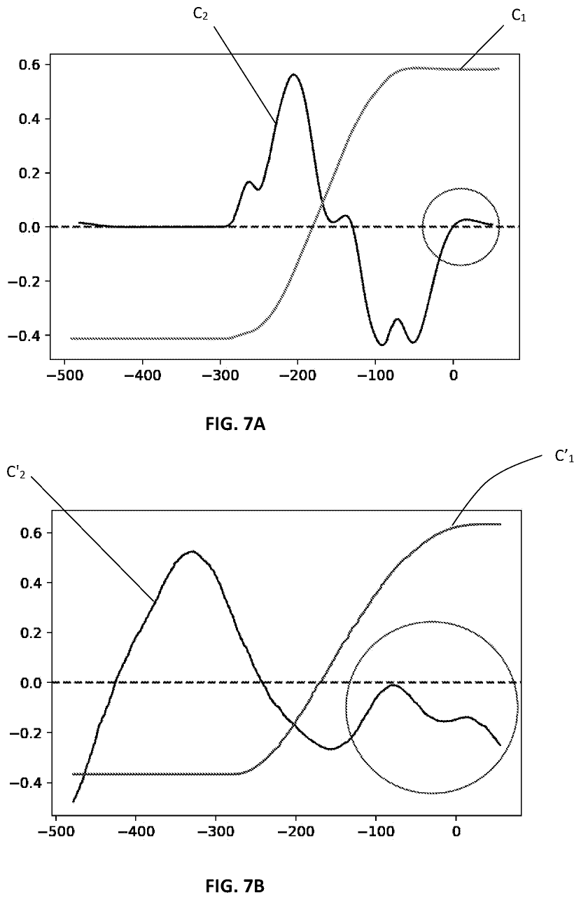

FIG. 8 illustrates an example of measuring/sensing motion patterns obtained from joystick sensors in a flight simulator. The pilot is required to maintain a certain height during the flight. Just before the pilot deviates from the required height (actual Error), a unique accelerations pattern emerges indicating error detection by the pilot's brain.

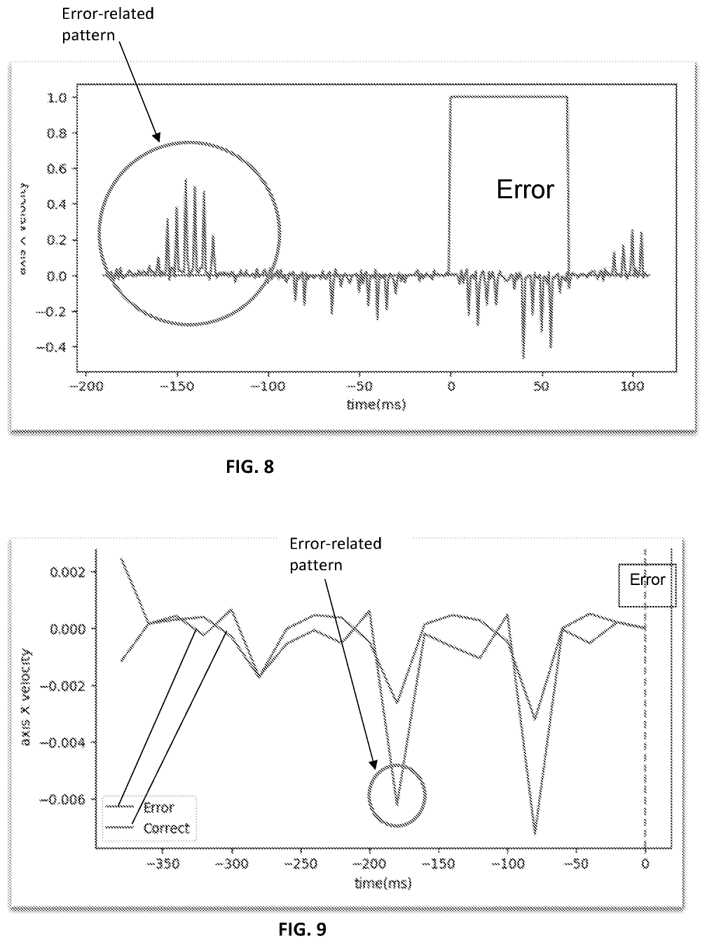

FIG. 9 illustrates an example of measuring/sensing motion patterns by gaming console (joystick) sensors while a game is being played. The gamer is required to avoid getting hit by an enemy spaceship gun. Just before the gamers spaceship takes an incorrect turn (actual Error) resulting in getting hit, a unique acceleration pattern (error-related pattern) emerges indicating error detection by the pilot's brain.

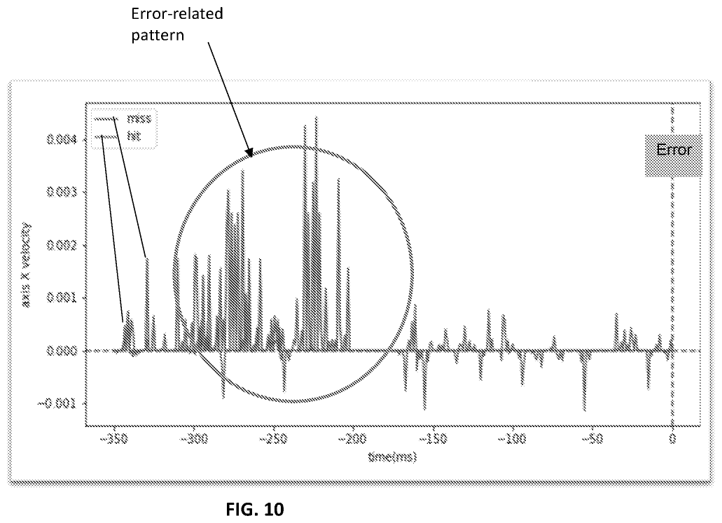

FIG. 10 illustrates an example of measuring/sensing motion patterns from joystick sensors in a flight simulator. The pilot is required to shoot a target. Just before the pilot shoots the target and misses it (actual Error), a unique accelerations pattern emerges indicating error detection.

As mentioned above, in some embodiments, the command data being measured/sensed may additionally to the motion command data include electromyography (EMG) data and/or electroencephalography (EEG) data. EMG provides information related to electrical activity produced by skeletal muscles participating in certain actions. The electric activity measured at a skeletal muscle which is involved in an action is different when erroneous action is performed as compared to non-erroneous (e.g. correct) actions. EEG provides recording of electrical activity along the scalp. EEG data measured during an erroneous command is different than the EEG data measured while a correct-command takes place.

In the lab when looking for EEG signals related to brain error detection, the search is locked to the incorrect key press (the search time-window is around the incorrect key press). Under lab conditions this manipulation is quite easy to perform because the measurement is done on short discrete responses and it is clear when the stimulus to which participants responded appears, when the response started, when the response ended and whether the response was correct or incorrect. However, when looking for signals related to error detection in the real world, in situations where the responses are prolonged and natural and not discrete, especially when the algorithm operates as a closed loop mechanism without access to external stimuli, and without access to response outcome (correct or incorrect) it is very difficult to know where to look for the error detection-related signal.

The solution proposed in the present invention is to lock the search to indications of error compensation or correction in the analyzed motion pattern. The rationale is that it is likely that an error detection occurred prior to the compensation or correction. Thus, when the data analysis algorithm is not running in a closed loop mode of operation, it can be entered with external information about manipulations that represent compensation or correction. For example, while driving, when the algorithm mainly relies on motion information received from steering wheel position data, additional information can be entered indicating activation of the accelerator pedal or brake. Alternatively, when the algorithm operates in a closed loop manner it can be taught to detect, in the motor output it analyzes, indications for error compensation or correction. For example, while driving, when the algorithm mainly relies on motor information received from steering wheel position data, it can be taught to detect, in the steering wheel position data, motor patterns indicating activation of the accelerator pedal or brake. The inventors have found that information about activation of the accelerator pedal or brake can be extracted from steering wheel position data.

In some cases, however, locking the search on the motion pattern segments corresponding to driver's brain activity aimed at error correction or compensation is too late in terms of capturing the appearance of the error-detection related motion patterns. In such cases the search is locked to the motion pattern segment(s) corresponding to an action that preceded the corrective or compensatory action.

Alternatively or additionally, the search can be locked to motion pattern segment(s) corresponding to an action that reflects a preparation for the corrective or compensatory action. For example, when monitoring the process of driving a car, locking the search to the time window around the release of the accelerator that precedes the pressing of the brake. This is because releasing the accelerator is an earlier indication than pressing the brake that the brain has detected an error. When collecting information from the movement of the hand on the steering wheel and non-activation of the pedals by the driver, the sensing data about the movement of the steering wheel can also be used. For example, when an overshoot is observed, the search can be locked to the corrective sub movement or to the ending section of the overshot itself where the brain probably attempts to arrest the overreaching motion.

Generally speaking, regardless of the type of sensing data (measured motion patterns and possibly also EEG and/or EMG data), the technique of the invention is aimed at differentiating incorrect actions from correct actions while overcoming individual differences in the movement profile. For example, an individual may exhibit a noisy movement characterized by irregular movement patterns especially around movement segments where error-detection related movement patterns are supposed to be found. To this end, the data analysis aimed at revealing the error-related movement pattern is a relative computation. More specifically, any anomalies or unique patterns found in an individual movement profile, are to be compared against its neighboring patterns. This means that even in a case where a certain pattern is determined as being indicative of an error based on a large sample of individual's motion, it still is to be compared against neighboring patterns in the individual movement profile.

Thus, according to the invention, the sensed/measured motion pattern data is analyzed to determine whether the movement progress deviates from the initial movement plan or goal. Indication of a goal directed movement can be based on several parameters, including: early differentiation, before movement completion; late differentiation based on indications at later stages of a movement; and possibly also an initial large motion (i.e., primary sub-movement) immediately followed by small motions (i.e., secondary sub-movement); relations between kinematic or EMG parameters of the relevant movement and kinematic or EMG parameters of the recovery phase of that movement.

As described above, the sensing system may include one or more sensors of any known suitable type capable of providing motion pattern over time of at least a part of individual's body, measured either directly from said body part of from an effect of the body part action on a certain device. Thus, the sensor(s) may or may not be directly connected to the device, but the measured motion command data may be measured directly from the body part of the user and the body part does not necessarily directly affect the operation of any device. For example, where reactions of a bodily system are monitored while the user performs a purposeful action while not operating any device at all or while a user performs a purposeful action aimed at controlling a non-electronic device and the monitored/measured values are used for detecting interacting-errors.

According to a specific example, this is so where an operator opens a door knob or when an operator hits a tennis ball with a racket. The sensor(s) may include a camera monitoring eye movement and/or changes in pupil diameter or eye blood vessels, the changes providing the relevant motion command data. Likewise, the sensor(s) may include a watch or bracelet strapped around the wrist of an individual and used for measuring skin electrical conductance, operating limbs kinematics or operating limbs EMG activity.

In some embodiments, the sensing data includes measurement of eye movements. Analysis of such sensing data may include identification of sub-movements of saccadic eye movements (under shoots and over shoots, drifts), a ratio between the saccadic movements of each eye. Specifically, during driving there is a challenge to identify eye movements related to monitoring the road conditions. To this end, information on road conditions can be obtained from the vehicle itself other vehicles or from cameras. Alternatively or additionally, the sensing data can be analyzed to identify one or more of the following: a rate of eye movement that characterizes tracking road conditions; a combination of eye movement with head movement or a distinction between eye movement and head movement (analysis of eye movements that are not accompanied by head movements or are accompanied by minimal head movements, or are not led by the head). Moreover, in the case of sub movements obtained from eye movement data, sub movements may occur along several dimensions and different angles: left or right, up or down and circular movements.

Additionally, a measurement of the coordination between hand movement and head movement or eye movement can provide important motion pattern data. For example, when cognitive ability deteriorates, the coordination between hand, head and eye movements decreases. There are more movements that are not coordinated with the others and the time that passes between eye movement and hand movement increases.

In some embodiments, a combination of different types of sensors (e.g. including both sensor(s) not connected to any device and sensor(s) connected to a device being operated by an individual) each measuring a different type of motion command data, can be used together.

In order to detect errors in the individual's activity, goal-directed movements are to be differentiated from motor reactions that have nothing to do with goal pursuit. Most motor maneuvers or reactions performed during daily activities are incidental representing no unconscious or conscious goal-directed behavior. Yet, even incidental motor maneuvers often involve error-related like motor anomalies resulting from factors such as mechanical, neural or muscular noise. Since such motor anomalies do not represent an error detection, such motor anomalies are to be excluded from further analysis. This is mostly apparent when the error-detection system is aimed at constantly monitoring individual's motor reactions during individual's daily activities because daily activities continuously involve incidental maneuvers.

Such challenge is absent from lab settings or other arranged settings where goal-directed movements can be easily differentiated from incidental maneuvers. For example, in a lab setting a case where an individual is required to aim a movement toward a target (goal-directed movement) is differentiated or compared to a case where an individual is required to produce spontaneous movements in the absence of target aiming instructions.

As described above, the analysis of the goal directed motion patterns utilizes a segmentation technique, based on early and later differentiations. This segmentation method allows for considerable reduction of the proportion of non-corrective sub-movements out of all analyzed sub-movements.

According to the known approaches, only corrective sub-movements are related with error detection in the human brain. However, differentiating these from non-corrective sub-movements is considered as almost impossible. The known techniques have shown that critical parameters allowing differentiation between the two types of sub-movements depend on task, type of movement, and speed of movement. Therefore, a given parameter may be suitable in one environment situation or task and completely irrelevant in another environment situation or task.

The segmentation method of the invention described above allows for data collection in various daily situations, while differentiating between events where the brain detects errors from events where the brain does not detect errors. Thus, across numerous ecological, daily situations, it facilitates the collection of parameters which are more prominent in ecological movements where error detection is active and absent from ecological movements where error detection is not active. Such segmentation method improves the ability for real time categorization of a certain movement or movement related pattern as erroneous and facilitate conclusions driven from error detection (e.g., individual's performance or state), through refinement of motion command data and motion command reference data.

Upon movement segmentation, a highly accurate technique for identifying corrective sub-movement is by analyzing sub-movements occurring at the last portion of a movement, i.e. slightly before and slightly after movement termination. Alternatively, within a movement, searching for sub-movements occurring slightly before and slightly after the peak of kinematic parameters, such as velocity, acceleration and so forth. The criterion for discerning movement segmentations occurring slightly before and slightly after movement termination can be based on time (i.e., 200 milliseconds before and after movement termination), or, because the profile of a movement is constructed of bursts or clusters of kinematics, it can be based on a number of clusters before and after movement termination (i.e., one cluster).

Additionally, the time gap between movement termination or movement kinematics peak and the sub-movement may indicate a corrective sub-movement. Alternatively, the ratio of initial movement kinematic parameter values to secondary movement kinematic parameter values may serve to indicate a corrective sub-movement. Usually the values of the secondary movement are smaller than the values of the initial movement.

It should be noted that, because in essence the principles described above are all related to the development of a motion or motion derivatives, the same principles can be applied using any type of sensors, such as pressure or force sensors.

Additionally, in order to differentiate a volitional, intended movement (e.g., a volitional kinematic change) from a corrective sub-movement (e.g., a completely or partially automatic kinematic change) the slope of the kinematic change at the last part of the terminated movement (may indicate response arrest) and the slope of the kinematic change at the first part of the new movement (may indicate a corrective response) can be calculated. Erroneous movements that need to be corrected are terminated faster than movements in which no corrective activity is needed. New corrective movements tend to develop faster than preplanned movements.

Additionally, in order to differentiate non-corrective sub-movements from corrective sub-movements, a comparison can be made between measured data indicating an error collected under conditions where a user is expected to exhibit highly functioning cognitive operational state and corresponding reference data indicating an error where a user is expected to exhibit reduced cognitive operational state (e.g., low motivation, intoxication, mental fatigue, drowsiness, stress, inattention, vertigo, motion sickness). It is assumed that only corrective sub-movements will systematically react to such comparison.

In order to differentiate motion patterns associated with error detection, compensation or correction from motion patterns associated with correct responses, especially when error detection indices are used to indicate a person's cognitive operational state, it will sometimes be necessary to adjust the error detection indices that indicate a normal or abnormal cognitive state, to the speed of movement of the vehicle in which the person is driving or to the speed of movement of the person himself. The reason is that sometimes the error detection relies on response outcome feedback or on environmental information indicating a need for response update. When the vehicle in which the person is driving or the person himself moves faster, the response outcome feedback or the response update que are available earlier.

For example, in order to differentiate a volitional, intended/goal directed movement (e.g., a volitional kinematic change) from a corrective sub-movement (e.g., a completely or partially automatic kinematic change indicating error detection) the slope of the kinematic change at the last part of the terminated movement and the slope of the kinematic change at the first part of the new movement can be determined. If the slope of the kinematic change at the last part of the terminated movement is significantly lower than the slope of the kinematic change at the first part of the new movement (i.e. descending curve of the motion pattern is shallower than the successive ascending curve), this is indicative of the corrective sub-movement. Once a corrective sub-movement is identified, the corresponding cognitive operational state of a person may be scored by the value of a ratio between the slope of the descending kinematic change at the last part of the terminated movement and the slope of the ascending kinematic change at the first part of the new movement.

This value may be adjusted for motion speed. If the person is operating a vehicle adjustment is to be made to vehicle speed. Alternatively, adjustment is to be made to movement speed (time from initiation to termination). In fact, every value indicating an error-related pattern or a pattern indicating a person cognitive operational state may be adjusted for motion speed. This is because sometimes the brain error detection mechanism relies on information received from the environment and the faster the vehicle or the person is, the sooner this information is received in the person's brain.

Sometimes the analysis that determines whether a particular movement pattern indicates an error detection or whether a particular movement pattern indicates a decrease in cognitive ability depends on the type of movement being analyzed. For example, in movements where there is no corrective submovements the ratio between the slope of the segment from the beginning of the motion to its maximum intensity (ascending curve) and the slope of the segment from the maximum intensity to the end of the movement (descending curve) is usually symmetrical. When a person's movement control or error control is not optimal as a result of cognitive decline, symmetry is impaired. In movements in which there is an overshoot corrective sub-movement only the descending curve should be calculated although an adjustment should be made for the speed of movement or the speed of the vehicle if the person was traveling in the vehicle. In movements in which there is an undershot corrective sub movement, the time from the beginning of the undershoot descending curve to the beginning of the immediate corrective submovement is calculated.

Another method for facilitation of collection of parameters which are more prominent in ecological movements where error detection is active and absent from ecological movements where error detection is not active is by relying on data received from machines which have access to individual movements and/or the outcomes of individual's movements (e.g., erroneous, correct). For example, modern cars and navigation systems are equipped with technology such as cameras, radars, motion detectors and GPS capturing, registering and alerting for driver's maneuvers. For example, a modern car can tell when a driver makes an error such as deviating from a lane and a navigation system can tell when a driver is making an error such as missing a turn. Other types of errors registered by cars are driver's emergency reactions to sudden environmental changes such as a sudden brake in response to brake lights illumination. These types of errors may yield different type of error-related motor patterns and so, data received from a machine, in this case, the car or the navigation system may facilitate the collection of parameters which are more prominent in one type of error than in the other. Additionally, as in the previous cases, such segmentation methods improve the ability for real time categorization of a certain movement or movement related pattern as erroneous and facilitate conclusions driven from error detection (i.e., user's performance or state), through refinement of motion command data and motion command reference data.

Motion command reference data values which enable to identify errors or motion command reference data values which enable to use individual's errors in order to identify individual's operational state can be prepared using various techniques. According to one example, various types of individual daily activities of a plurality of individuals are monitored, and a corresponding plurality of motion command data pieces are recorded in association with each activity type. Motion command data statistics (e.g. average and standard deviation) of the recorded data can be calculated to serve as motion command reference data to be used for error identification or motion command reference data values which enable to use individual's errors in order to identify individual's operational state with respect to respective activities. The calculated motion command data statistics may include the error-relating reference data and reference data indicative of correct motion command and/or correct action.

Motion command data being measured may be recorded (according to various recording policies) for a certain period of time or until a certain desired amount of data is recoded before it is used for calculating the statistics. Motion command data may be continuously recorded during certain activity and used for enhancing the motion command data statistics.

Motion command data statistics may be calculated for various specific setups such as: a specific individual, a specific group of individuals, specific types of activities, etc.

Motion command data may be recorded and stored during the activity of a single individual, allowing calculation of individual-specific motion command data statistics providing a personalized characterization of the individual's performance. Additionally, or alternatively, motion command data can be recorded during the specific activity of many different individuals, thus enabling to obtain normal motion command reference data representing the distribution of motion command data values in a monitored population of individuals. Normal motion command reference data can be used for calculating normal motion command data statistics, including for example the average and standard deviation of the motion command data values collected from the population of individuals.

Both user-specific and population-specific motion command reference data may be calculated by a statistics module, which may be part of the control system 12 or of the external control station 16 being in data communication with the control system 12 to receive the recorded motion command data from the control system 12 and similar control systems associated with a plurality of individuals. The data is consolidated and stored at the control station 16 which calculates the motion command data statistics.

The obtained motion command data statistics can thus be provided at the control system 12 (e.g. from the control station 16 where such data is calculated). The control system 12 may use this statistics data during real-time monitoring of the daily activities of a respective individual for identifying errors (erroneous actions).

Motion command reference data can be enhanced by additional data, including for example motion command data collected while an actual error is performed and corrected. A correction of an action which is made by the individual provides an explicit indication that the action was erroneous. The motion command data which is recorded during such a corroborated error can be used for obtaining additional information indicative of specific motion command data values which characterize an erroneous action. Likewise, motion command data recorded during a correct action can be used for obtaining additional information indicative of motion command data values which characterize a correct action.

Corroboration of motion command reference data can be obtained for example by prompting an individual after an action is performed, asking the individual whether the performed action was erroneous or not and/or by monitoring spontaneous individual correction of actions and/or manual or voice gestures indicating an error.

1. A monitoring system for monitoring an individual's activity, the monitoring system comprising a control system configured as a computer system comprising data input and output utilities, a memory, and a data processor and analyzer, the control system being configured and operable to be responsive to input data comprising sensing data collected over time from at least a part of individual's body by one or more sensors of predetermined one or more types and being indicative of a motion pattern characterizing a certain activity of the individual, to process said input data by applying thereto at least one machine learning model and generate output data indicative of a cognitive error detection by said individual in said activity characterizing a cognitive operational state of the individual during said activity.

2. The monitoring system according to claim 1, wherein the control system is configured and operable for data communication with one or more measured data providers to receive, from each measured data provider, the input data comprising the sensing data.

3. The monitoring system according to claim 1, wherein the input data comprises at least one of the following:
(i) the input data further comprises data indicative of said one or more types of the sensors collecting said sensing data;
(ii) the input data comprises data indicative of said activity performed by the individual;
(iii) the input data comprises the sensing data comprising said motion patterns measured over time on a device operated by the individual during said activity; and
(iv) the input data further comprises electroencephalography (EEG) data measured at the individual's brain, and/or electromyography (EMG) data measured at a skeletal muscle of the at least one body part of the individual.

4. (canceled)

5. The monitoring system according to claim 1, wherein said sensing data comprises said motion patterns measured over time and being indicative of movement intention or movement of said at least part of the individual's body during said activity.

6. (canceled)

7. The monitoring system according to claim 1, wherein the data processor and analyzer is configured and operable to carry out the following:
analyze the motion patterns to determine movement progress deviation from an initial movement goal,
identify and analyze different types of deviations indicating error detection in the individual's brain, and compare identified error detection related deviations to predefined reference error detection related deviations, to thereby determine whether at least one of the identified error related deviations and a relation between identified and reference error related deviations is indicative of a change in the individual's cognitive operational state.

8. The monitoring system according to claim 7, wherein said relation is a difference value between the identified and reference error related deviations.

9. The monitoring system according to claim 1, wherein the data processor and analyzer comprises:
a sensing data analyzer configured and operable to analyze the input data, and, upon identifying that the motion patterns comprise a pattern corresponding to a goal directed movement performed by the individual during said activity, generating corresponding decision data;
an error identifier utility configured and operable to identify at least one predetermined segment in the goal directed movement pattern, and process said at least one predetermined segment, and, upon identifying in said at least one predetermined segment motion profile indicative of movements cognitively recognizable by the individual as error-related movements, generating output data indicative of error-related motion pattern enabling evaluation of the operational state of the individual.

10. The monitoring system according to claim 9, characterized by at least one of the following: (a) the at least one predetermined segment of the motion pattern includes motion from a first instant of the goal-directed movement until after a first instant of a latest motion that served the decision of the goal-directed movement; (b) the error identifier utility is configured and operable to apply said at least one machine learning model to the goal directed movement pattern to identify said at least one predetermined segment and lock a search on said at least one predetermined segment for one or more characteristic features with respect to the activity being performed by the individual and the one or more predetermined types of sensors providing the sensing data; and (c) the error identifier utility is configured and operable to apply machine learning based processing to the goal directed movement pattern associated with said certain individual's activity and being collected over time by the sensor of the predetermined type.

11. (canceled)

12. The monitoring system according to claim 9, wherein the error identifier utility is configured and operable to apply machine learning based processing to the goal directed movement pattern associated with said certain individual's activity and being collected over time by the sensor of the predetermined type by carrying out one of the following:
(1) sorting movements forming said motion pattern into correct movements and incorrect movements; identifying differences in features of the correct movements and the incorrect movements to define one or more characteristic feature uniquely characterizing errors; and
determining a change in said one or more characteristic features resulting from a change in the individual's cognitive operational state, in association with each of one or more factors affecting the cognitive operational state of the individual; and
(2) identifying in said motion pattern movements having different features; selecting one or more characteristic features from the movement located at extreme values of normally distributed motion pattern; and determining a change in said one or more characteristic features resulting from a change in the individual's cognitive operational state, in association with each of one or more factors affecting the cognitive operational state of the individual.

13. (canceled)

14. The monitoring system according to claim 9, wherein the error identifier utility is configured and operable to apply said at least one machine learning model to the goal directed movement pattern to identify said at least one predetermined segment and lock a search on said at least one predetermined segment for one or more characteristic features with respect to the activity being performed by the individual and the one or more predetermined types of sensors providing the sensing data, said one or more characteristic features including one or more of the following: submovements appearance in time relation to movement kinematics peak, submovements appearance in time relation to movement kinematics termination, a ratio of an ascending slope of at least a part of movement to a descending slope of at least a part of movement's, a ratio between different parts of an ascending slope of movement, a ratio between different parts of a descending slope of movement, a temporal frequency of velocity derivatives' changes, similarity of velocity derivatives changes across the time segment, temporal frequency of submovements, a time pattern of slopes of the kinematic change of movement being terminated followed by beginning of a successive movement.

15. The monitoring system according to claim 9, characterized by at least one of the following:
the control system further comprises a detector utility configured and operable to analyze the error-related motion pattern and generate operational state data characterizing the operational state of the individual;
the error identifier utility is configured and operable to perform said processing of the at least one predetermined segment by analyzing motion command data of the individual's brain resulting in said motion pattern over corresponding reference motion command data, and determine error-related motion command data originated in the individual's brain;
said sensing data analyzer is configured and operable to analyze movement kinematic data derived from said motion patterns to determine differentiation between movements that can be classified as goal directed and non goal directed, said differentiations comprising at least one of the following: early differentiation, before movement completion, and late differentiation based on later stages of the movement;
said analyzer is configured and operable to analyze the motion patterns and upon identifying therein primary sub-movements corresponding to an initial relatively large motion immediately followed by secondary sub-movements corresponding to relatively small motion, classifying the motion pattern as corresponding to the goal directed movement; and
said at least one predetermined motion pattern segment includes successive segments indicative of, respectively, initiation of the goal-directed movement, undershoot and overshoot corrective sub-movements, and undershoot corrective sub-movement immediately before movement termination.

16. The monitoring system according to claim 9, wherein the error identifier utility is configured and operable to perform said processing of the at least one predetermined segment by analyzing motion command data of the individual's brain resulting in said motion pattern over corresponding reference motion command data, and determine error-related motion command data originated in the individual's brain, the error identifier utility being configured and operable to perform said processing of the predetermined segments by analyzing motion command data of the individual's brain resulting in said motion pattern over corresponding reference motion command data, and determine error-related motion command data originated in the individual's brain; and the detector utility is configured and operable to analyze error-related motion command data resulting in the error-related motion pattern over corresponding motion command error-related reference data.

17. (canceled)

18. The monitoring system according to claim 9, wherein said sensing data analyzer is configured and operable to analyze movement kinematic data derived from said motion patterns to determine differentiation between movements that can be classified as goal directed and non goal directed, said differentiations comprising at least one of the following: early differentiation, before movement completion, and late differentiation based on later stages of the movement, the determination of said early differentiation comprising analyzing the movement kinematics and determining a rate of movement kinematics development from a first measured motion command data value to movement kinematics maximum.

19. (canceled)

20. The monitoring system according to claim 18, wherein the determination of said early differentiations comprises:
analyzing the movement kinematics starting from a first measured motion command data value originated in the individual's brain at a first instant along the motion pattern of executed command,
based on the first measured motion command data value, determining an expected motion command data value for a later instant along the executed command,
comparing measured motion command data value at the later instant with said expected motion command data value, and upon identifying that a difference between the expected motion command data value and the measured motion command data value complies with a predefined criterion, classifying the motion pattern as corresponding to the goal directed movement.

21. (canceled)

22. (canceled)

23. The monitoring system according to claim 1, wherein the control system is further configured and operable for carrying out at least one of the following: recording data indicative of the cognitive error detection characterizing the cognitive operational state of the individual during said activity, thereby enabling use of the recorded data for optimizing corresponding error-related reference data; and generating notification data indicative of the cognitive operational state of the individual during said activity.

24. (canceled)

25. (canceled)

26. The monitoring system according to claim 1, wherein the one or more sensors comprise at least one accelerometer.

27. (canceled)

28. An electronic device comprising: a sensing system including one or more sensors of predetermined one or more types configured and operable to provide sensing data including motion pattern measured over time from at least a part of individual's body during an individual's activity and being indicative of motion characterizing the individual's activity; and the monitoring system according to claim 1.

29. A method for monitoring an individual's activity, the method being carried out by a computerized system being in data communication with one or more measured data providers, the method comprising:
receiving, from the measured data provider, input data comprising motion patterns measured over time from at least a part of individual's body by a sensing system comprising one or more sensors of predetermined one or more types and being indicative of motion characterizing the individual's activity;
processing said input data and generating output data indicative of a cognitive error detection by said individual in said activity characterizing a cognitive operational state of the individual during said activity, said processing comprising:
analyzing the motion patterns, and, upon identifying that the motion patterns comprise a pattern corresponding to a goal directed movement performed by the individual during said activity, generating corresponding decision data;
processing the goal directed motion pattern by applying thereto at least one machine learning model to identify one or more predetermined segments in the goal directed movement pattern, and process said predetermined segments to identify whether said one or more predetermined segments are indicative of movements cognitively recognizable by the individual as error-related movements, and upon identifying data indicative of the movements cognitively recognizable by the individual as error-related movements, generating output data indicative of error-related motion pattern enabling evaluation of the operational state of the individual.

30. The method according to claim 29, characterized by at least one of the following:
further comprising analyzing the error-related motion pattern and generating operational state data characterizing the operational state of the individual;
the predetermined segments of the motion pattern include motion pattern segments from a first instant of the goal-directed movement until after a first instant of a latest motion pattern that served the decision of the goal-directed movement;
said processing of the predetermined segments comprises analyzing motion command data of the individual's brain resulting in said motion pattern over corresponding reference motion command data, and determine error-related motion command data originated in the individual's brain; and
said processing of the goal directed motion pattern, associated with said certain individual's activity and being collected over time by the sensor of the predetermined type.

31. (canceled)

32. The method according to claim 29, wherein said processing of the predetermined segments comprises analyzing motion command data of the individual's brain resulting in said motion pattern over corresponding reference motion command data, and determine error-related motion command data originated in the individual's brain, the method further comprising analyzing the error-related motion command data resulting in the error-related motion pattern over corresponding motion command error-related reference data.

33. (canceled)

34. The method according to claim 29, wherein said processing of the goal directed motion pattern, associated with said certain individual's activity and being collected over time by the sensor of the predetermined type, by applying thereto at least one machine learning model comprises one of the following:
sorting movements forming said motion pattern into correct movements and incorrect movements; identifying differences in features of the correct movements and the incorrect movements to define one or more characteristic feature uniquely characterizing errors; and determining a change in said one or more characteristic features resulting from a change in the individual's cognitive operational state, in association with each of one or more factors affecting the cognitive operational state of the individual; and
identifying in said motion pattern movements having different features; selecting one or more characteristic features from the movement located at extreme values of normally distributed motion pattern; and determining a change in said one or more characteristic features resulting from a change in the individual's cognitive operational state, in association with each of one or more factors affecting the cognitive operational state of the individual.

35. (canceled)

36. A non-transitory program storage device readable by a computer, tangibly embodying a program of instructions executable by the computer to perform a method of detection of movements cognitively recognizable by an individual as error-related movements, the method comprising:
obtaining input data comprising motion patterns measured over time from at least a part of individual's body by one or more sensors of predetermined one or more types and being indicative of motion characterizing a certain activity of an individual;
analyzing the motion patterns, and, upon identifying that the motion patterns comprise a pattern corresponding to a goal directed movement performed by the individual during said activity, generating corresponding decision data;
identifying predetermined segments in the goal directed movement pattern, and processing said predetermined segments, and, upon determining that said predetermined segments are indicative of movements cognitively recognizable by the individual as error-related movements, generating output data indicative of error-related motion pattern enabling evaluation of the operational state of the individual.

37. A computer program product comprising a non-transitory computer useable medium having computer readable program code embodied therein for detection of movements cognitively recognizable by an individual as error-related movements, the computer program product comprising:
computer readable program code for causing the computer to obtain input data comprising motion patterns measured over time from at least a part of individual's body by one or more sensors of predetermined one or more types and being indicative of motion characterizing a certain activity of an individual;
computer readable program code for causing the computer to analyze the motion patterns, and, upon identifying that the motion patterns comprise a pattern corresponding to a goal directed movement performed by the individual during said activity, generating corresponding decision data; and
computer readable program code for causing the computer to identify predetermined segments in the goal directed movement pattern, and process said predetermined segments, and, upon determining that said predetermined segments are indicative of movements cognitively recognizable by the individual as error-related movements, generate output data indicative of error-related motion pattern indicative of the operational state of the individual.
